(19) United States
(12) Patent Application Publication
(10) Pub. No.: US 2026/0162820 A1
(43) Pub. Date: Jun. 11, 2026

(54) DISTRIBUTED ARCHITECTURE FOR PATIENT MONITORING SYSTEM FOR GENERATING PATIENT ALARMS

(71) Applicant: Masimo Corporation, Irvine, CA (US)

(72) Inventor: Ammar Al-Ali, San Juan Capistrano, CA (US)

(21) Appl. No.: 19/356,618

(22) Filed: Oct. 13, 2025

Related U.S. Application Data
(60) Provisional application No. 63/707,639, filed on Oct. 15, 2024.

(51) Int. Cl. G16H 40/67; G16H 10/60; G16H 15/00; G16H 50/20; G16H 70/60; G16H 80/00

(52) U.S. Cl. CPC G16H 40/67; G16H 10/60; G16H 15/00; G16H 50/20; G16H 70/60; G16H 80/00

(57) ABSTRACT

A physiological monitoring system can monitor health status of a subject having a wireless wearable device. The monitoring system can have distributed monitoring devices in communication with each other and a remote server. A monitoring hub of the system can establish wireless communication with the wearable device to wirelessly receive physiological data from the wearable device. The monitoring hub can transmit the physiological data to the remote server without processing the physiological data for centralized processing by the server. The server can generate patient alarms and cause the alarms to be output by the distributed monitoring devices. In instances when connection to the server is interrupted or lost, the monitoring hub and/or wearable device can continue, without interruption, monitoring the subject continuously and in real-time, such as for triggering of alarm conditions, until connection with the server is improved or reestablished.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD

The present disclosure relates to physiological monitoring devices, systems, and methods.

### BACKGROUND

Hospitals, nursing homes, and other patient care facilities typically utilize a number of sensors, devices, and/or monitors to collect or analyze a patient's physiological parameters. Various conventional sensor systems exist which collect physiological data using physiological sensors, process the data, and display the data on a display device. Clinicians, including doctors, nurses, and other medical personnel, use the physiological parameters obtained from patient monitors to diagnose illnesses and to prescribe treatments. Clinicians also use the physiological parameters to monitor patients during various clinical situations to determine whether to increase the level of medical care given to patients.

### SUMMARY

Many hospitals, nursing homes, and other patient care facilities utilize remote servers as central processing computing systems that process sensor data from distributed computing devices, such as sensors, monitoring hubs, user devices, other servers, and the like. Use of remote servers, such as a local cloud, can reduce processing requirements on the distributed devices. However, reliance on computing systems such as servers for continuous and/or real-time data processing needs without capability to continue such physiological monitoring when communication connection to the servers is interrupted or lost can result in disrupted patient care, loss of physiological data (such as during a time in which sensors are not connected to the servers), and delay in detecting and attending to adverse events.

Various implementations of systems, methods, and devices within the scope of the appended claims each have several aspects, no single one of which is solely responsible for the desirable attributes described herein. Without limiting the scope of the appended claims, the description below describes some prominent features.

Details of one or more implementations of the subject matter described in this specification are set forth in the accompanying drawings and the description below. Other features, aspects, and advantages will become apparent from the description, the drawings, and the claims. Note that relative dimensions of the following figures may not be drawn to scale.

In some aspects, the techniques described herein relate to a physiological monitoring system: a remote device including one or more hardware processors configured to: receive physiological data from a noninvasive medical monitoring device; process the physiological data using a first alarm algorithm based on a plurality of physiological inputs to determine whether a first alarm condition is satisfied; generate and transmit an alarm to the noninvasive medical monitoring device if the first alarm condition is satisfied; and transmit alarm configuration data specifying at least one second alarm condition to the noninvasive medical monitoring device; and the noninvasive medical monitoring device including one or more hardware processors in communication with a physiological sensor and a communication component, the one or more hardware processors configured to: store the alarm configuration data received from the remote device in local memory; determine whether a connection with the remote device is lost; responsive to a determination that the connection with the remote device is lost, process physiological data using a second alarm algorithm based on the stored alarm configuration data to determine whether the second alarm condition is satisfied, and generate a local alarm only if the connection with the remote device is lost and the second alarm condition is satisfied; wherein the second alarm algorithm is less computationally intensive than the first alarm algorithm, and wherein, when the connection with the remote device is not lost, the noninvasive medical monitoring device is configured to rely on the remote device to generate alarms.

In some aspects, the techniques described herein relate to a system, wherein the first alarm algorithm executed by the remote device includes analysis of multiple physiological parameters, historical trends, or contextual patient information.

In some aspects, the techniques described herein relate to a system, wherein the second alarm algorithm executed by the noninvasive medical monitoring device includes comparison of a physiological parameter to a predetermined threshold.

In some aspects, the techniques described herein relate to a system, wherein the noninvasive medical monitoring device is further configured to buffer physiological data generated during a period in which the connection with the remote device is lost, and to transmit the buffered physiological data to the remote device when the connection is reestablished.

In some aspects, the techniques described herein relate to a system, wherein the remote device is further configured to update the alarm configuration data and transmit updated alarm configuration data to the noninvasive medical monitoring device.

In some aspects, the techniques described herein relate to a system, wherein the first alarm algorithm executed by the remote device determines the first alarm condition based on a combination of a threshold or threshold range for a physiological parameter and at least one of: patient measurement history, patient comorbidity indications, measurements from other physiological parameters, types of procedures occurring, treatments administered or indicated, or other risk factors.

In some aspects, the techniques described herein relate to a system, wherein the alarm configuration data transmitted from the remote device to the noninvasive medical monitoring device includes at least one of: a threshold value, a threshold range, a time duration, a rate of change, or a combination thereof.

In some aspects, the techniques described herein relate to a system, wherein the noninvasive medical monitoring device is further configured to output the local alarm as at least one of a visual, auditory, or haptic signal.

In some aspects, the techniques described herein relate to a system, wherein the determination that the connection with the remote device is lost is based on at least one of: a signal strength condition, a failure to receive an acknowledgement signal, or a timeout period without receiving wireless signals from the remote device.

In some aspects, the techniques described herein relate to a system, wherein the noninvasive medical monitoring device is further configured to receive updated alarm configuration data from the remote device and to overwrite a previously stored alarm configuration data in local memory.

In some aspects, the techniques described herein relate to a system, wherein the remote device is further configured to, upon reestablishing communication with the noninvasive medical monitoring device, receive buffered physiological data and reprocess the buffered data using the first alarm algorithm to determine whether any first alarm conditions would have been satisfied during a period of lost connection.

In some aspects, the techniques described herein relate to a system, wherein the noninvasive medical monitoring device is further configured to store a log of all local alarms generated during a period of lost connection and to transmit the log to the remote device when the connection is reestablished.

In some aspects, the techniques described herein relate to a system, wherein the remote device is further configured to generate a report indicating any discrepancies between alarms generated by the noninvasive medical monitoring device during a period of lost connection and alarms that would have been generated by the first alarm algorithm.

In some aspects, the techniques described herein relate to a system, wherein the noninvasive medical monitoring device is further configured to display an indication of connection status with the remote device.

In some aspects, the techniques described herein relate to a system, wherein the alarm configuration data includes patient-specific alarm thresholds determined by the remote device based on analysis of historical physiological data.

In some aspects, the techniques described herein relate to a noninvasive medical monitoring device, the medical device including: one or more hardware processors in communication with a communication component, the communication component configured to operate one or more wireless communication protocols, the communication component configured to permit wireless connection between the monitoring device and a remote device such that the monitoring device sends and receives, to and from the remote device, wireless signals; the one or more hardware processors in communication with a physiological sensor; and the one or more hardware processors configured to: determine whether the connection with the remote device is lost; responsive to a determination that the connection with the remote device is lost, determine at least one physiological parameter based on the at least one physiological signal; determine whether the physiological parameter satisfies a predetermined alarm condition; and responsive to the determination that the physiological parameter satisfies the predetermined alarm condition, generate an alarm.

In some aspects, the techniques described herein relate to a noninvasive medical monitoring device, wherein the alarm is normally generated by the remote device.

In some aspects, the techniques described herein relate to a noninvasive medical monitoring device, wherein the one or more hardware processors are configured to generate the alarm only if connection to the remote device is lost.

In some aspects, the techniques described herein relate to a noninvasive medical monitoring device, wherein the remote device includes one or more hardware processors configured to determine a physiological alarm condition based at least in part on a plurality of physiological inputs.

In some aspects, the techniques described herein relate to a noninvasive medical monitoring device, wherein the predetermined alarm condition and the determined alarm condition are different such that the monitoring device and remote device generate alarms under different physiological conditions associated with the physiological parameter.

In some aspects, the techniques described herein relate to a noninvasive medical monitoring device, wherein the one or more hardware processors are configured to receive, from the remote device, alarm configuration data that specifies the predetermined alarm condition that is used by the monitoring device for generating the alarm if connection to the remote device is lost.

In some aspects, the techniques described herein relate to a noninvasive medical monitoring device, wherein the one or more hardware processors are further configured to output the alarm to a display of the monitoring device.

In some aspects, the techniques described herein relate to a noninvasive medical monitoring device, wherein the one or more hardware processors are further configured to output the alarm to a display of a display terminal.

In some aspects, the techniques described herein relate to a noninvasive medical monitoring device, wherein the one or more hardware processors are further configured to output the alarm to a loudspeaker.

In some aspects, the techniques described herein relate to a noninvasive medical monitoring device, wherein the one or more hardware processors are configured to determine whether the connection with the remote device is lost based on a determination of whether a strength of the wireless signals satisfies a signal strength condition.

In some aspects, the techniques described herein relate to a noninvasive medical monitoring device, wherein the one or more hardware processors are configured to determine whether the connection with the remote device is lost based on a determination of whether an acknowledgement signal has been received from the remote device.

In some aspects, the techniques described herein relate to a noninvasive medical monitoring device, wherein the one or more hardware processors are configured to determine whether the connection with the remote device is lost based on a determination of whether the monitoring device has received, from the remote device, wireless signals within a timeout period.

In some aspects, the techniques described herein relate to a noninvasive medical monitoring device, wherein the generated alarm includes visual, auditory, or haptic feedback.

In some aspects, the techniques described herein relate to a noninvasive medical monitoring device, wherein the predetermined alarm condition is stored in local memory of the monitoring device.

In some aspects, the techniques described herein relate to a noninvasive medical monitoring device, wherein physiological data associated with the at least one physiological signal is stored in a buffer until connection with the remote device is reestablished.

In some aspects, the techniques described herein relate to a method for providing continuous care to a subject, the method including: under control of one or more hardware processors, the one or more hardware processors in communication with a communication component configured to permit wireless connection between a noninvasive medical monitoring device and a remote device, and the one or more hardware processors in communication with a physiological sensor having a plurality of LEDs configured to emit light of one or more wavelengths at a measurement site of a subject and at least one detector configured to generate at least one physiological signal associated with the one or more light wavelengths after attenuation by tissue at the measurement site of the subject: receiving the at least one physiological signal from the physiological sensor, the physiological signal indicative of a physiological parameter; establishing wireless connection between the monitoring device and the remote device; determining whether the connection with the remote device is lost; responsive to determining that the connection with the remote device is lost, determining the physiological parameter based on the at least one physiological signal; determining whether the physiological parameter satisfies a predetermined alarm condition; and responsive to determining that the physiological parameter satisfies the predetermined alarm condition, generating an alarm for output by the noninvasive medical monitoring device.

In some aspects, the techniques described herein relate to a method, wherein the alarm is normally generated by the remote device.

In some aspects, the techniques described herein relate to a method, wherein generating the alarm further includes generating the alarm only if connection to the remote device is lost.

In some aspects, the techniques described herein relate to a method, the method further including: determining, by the remote device, a physiological alarm condition based at least in part on a plurality of physiological inputs.

In some aspects, the techniques described herein relate to a method, wherein the predetermined alarm condition and the determined alarm condition are different such that the monitoring device and the remote device generate alarms under different physiological conditions associated with the physiological parameter.

In some aspects, the techniques described herein relate to a method, the method further including: under control of the one or more hardware processors, receiving, from the remote device, alarm configuration data that specifies the predetermined alarm condition that is used by the monitoring device for generating the alarm if connection to the remote device is lost.

In some aspects, the techniques described herein relate to a method, wherein generating the alarm further includes outputting the alarm to a display of the monitoring device.

In some aspects, the techniques described herein relate to a method, wherein generating the alarm further includes outputting the alarm to a display of a display terminal.

In some aspects, the techniques described herein relate to a method, wherein generating the alarm further includes outputting the alarm to a loudspeaker.

In some aspects, the techniques described herein relate to a method, wherein determining whether the connection with the remote device is lost further includes determining whether a strength of the wireless signals satisfies a signal strength condition.

In some aspects, the techniques described herein relate to a method, wherein determining whether the connection with the remote device is lost further includes determining whether an acknowledgement signal has been received from the remote device.

In some aspects, the techniques described herein relate to a method, wherein determining whether the connection with the remote device is lost further includes determining whether the monitoring device has received, from the remote device, wireless signals within a timeout period.

In some aspects, the techniques described herein relate to a method, wherein the generated alarm includes visual, auditory, or haptic feedback.

In some aspects, the techniques described herein relate to a method, wherein the predetermined alarm condition is stored in local memory of the monitoring device.

In some aspects, the techniques described herein relate to a method, wherein physiological data associated with the at least one physiological signal is stored in a buffer until connection with the remote device is reestablished.

In some aspects, the techniques described herein relate to a method for providing continuous care to a subject, the method including: receiving at least on physiological signal originating from a physiological sensor, the physiological signal indicative of a physiological parameter of a subject; establishing wireless connection between a noninvasive medical monitoring device and a remote device; determining whether the connection with the remote device is lost; responsive to determining that the connection with the remote device is lost, determining the physiological parameter of the subject based on the at least one physiological signal; determining whether the physiological parameter satisfies a predetermined alarm condition; and responsive to determining that the physiological parameter satisfies the predetermined alarm condition, generating an alarm.

In some aspects, the techniques described herein relate to a method, wherein the alarm is normally generated by the remote device.

In some aspects, the techniques described herein relate to a method, wherein generating the alarm further includes generating the alarm only if connection to the remote device is lost.

In some aspects, the techniques described herein relate to a method, the method further including: determining, by the remote device, a physiological alarm condition based at least in part on a plurality of physiological inputs.

In some aspects, the techniques described herein relate to a method, wherein the predetermined alarm condition and the determined alarm condition are different such that the monitoring device and the remote device generate alarms under different physiological conditions associated with the physiological parameter.

In some aspects, the techniques described herein relate to a method, the method further including: receiving, from the remote device, alarm configuration data that specifies the predetermined alarm condition that is used by the monitoring device for generating the alarm if connection to the remote device is lost.

In some aspects, the techniques described herein relate to a method, wherein generating the alarm further includes outputting the alarm to a display of the monitoring device.

In some aspects, the techniques described herein relate to a method, wherein generating the alarm further includes outputting the alarm to a display of a display terminal.

In some aspects, the techniques described herein relate to a method, wherein generating the alarm further includes outputting the alarm to a loudspeaker.

In some aspects, the techniques described herein relate to a method, wherein determining whether the connection with the remote device is lost further includes determining whether a strength of the wireless signals satisfies a signal strength condition.

In some aspects, the techniques described herein relate to a method, wherein determining whether the connection with the remote device is lost further includes determining whether an acknowledgement signal has been received from the remote device.

In some aspects, the techniques described herein relate to a method, wherein determining whether the connection with the remote device is lost further includes determining whether the monitoring device has received, from the remote device, wireless signals within a timeout period.

In some aspects, the techniques described herein relate to a method, wherein the generated alarm includes visual, auditory, or haptic feedback.

In some aspects, the techniques described herein relate to a method, wherein the predetermined alarm condition is stored in local memory of the monitoring device.

In some aspects, the techniques described herein relate to a method, wherein physiological data associated with the at least one physiological signal is stored in a buffer until connection with the remote device is reestablished.

In some aspects, the techniques described herein relate to a remote server for a physiological monitoring system, the server including one or more hardware processors configured to: receive physiological data from a noninvasive medical monitoring device; process the physiological data using a complex alarm algorithm based on a plurality of physiological inputs to determine whether a complex alarm condition is satisfied; generate and transmit an alarm to the noninvasive medical monitoring device if the complex alarm condition is satisfied; transmit alarm configuration data specifying at least one simple alarm condition to the noninvasive medical monitoring device for local use during connection loss; detect when communication with the noninvasive medical monitoring device is reestablished after a period of lost connection; receive buffered physiological data from the noninvasive medical monitoring device corresponding to the period of lost connection; backfill records of the server with the buffered physiological data; reprocess the backfilled physiological data using the complex alarm algorithm to determine whether any complex alarm conditions would have been satisfied during the period of lost connection; and, if so, generate and transmit retrospective alarms or notifications based on the reprocessed data.

In some aspects, the techniques described herein relate to a server, wherein the server is further configured to update patient records or logs based on the backfilled physiological data.

In some aspects, the techniques described herein relate to a server, wherein the server is further configured to generate a report indicating any discrepancies between alarms generated locally by the noninvasive medical monitoring device and alarms that would have been generated by the complex alarm algorithm during the period of lost connection.

In some aspects, the techniques described herein relate to a server, wherein the server is further configured to transmit updated alarm configuration data to the noninvasive medical monitoring device based on analysis of the backfilled physiological data.

In some aspects, the techniques described herein relate to a server, wherein the server is further configured to generate a notification to a clinician or user device if a retrospective alarm is generated based on the reprocessed buffered physiological data.

In some aspects, the techniques described herein relate to a server, wherein the server is further configured to annotate a patient record to indicate which physiological data was received in real time and which was backfilled after a period of lost connection.

In some aspects, the techniques described herein relate to a server, wherein the server is further configured to compare timestamps of buffered physiological data with server time to identify and flag any data gaps or inconsistencies.

In some aspects, the techniques described herein relate to a server, wherein the server is further configured to prioritize processing of backfilled physiological data upon reestablishment of communication with the noninvasive medical monitoring device.

In some aspects, the techniques described herein relate to a server, wherein the server is further configured to store a record of all periods of lost connection, including start and end times, and associate each period with the corresponding buffered physiological data.

In some aspects, the techniques described herein relate to a server, wherein the server is further configured to generate a summary report for each period of lost connection, the report including at least one of: a duration of the lost connection, a number of alarms generated locally by the noninvasive medical monitoring device, and a number of retrospective alarms generated by the server.

In some aspects, the techniques described herein relate to a server, wherein the server is further configured to transmit a confirmation to the noninvasive medical monitoring device upon successful receipt and processing of the buffered physiological data.

In some aspects, the techniques described herein relate to a server, wherein the server is further configured to update alarm configuration data for the noninvasive medical monitoring device based on trends or anomalies detected in the backfilled physiological data.

In some aspects, the techniques described herein relate to a server, wherein the server is further configured to provide a user interface displaying both real-time and backfilled physiological data, with visual indicators distinguishing between the two.

In some aspects, the techniques described herein relate to a server, wherein the server is further configured to generate an audit log of all alarms, including those generated in real time, those generated locally by the noninvasive medical monitoring device, and those generated retrospectively by the server.

In some aspects, the techniques described herein relate to a server, wherein the server is further configured to automatically adjust parameters of the complex alarm algorithm based on analysis of the backfilled physiological data.

In some aspects, the techniques described herein relate to a server, wherein the server is further configured to synchronize time stamps between the server and the noninvasive medical monitoring device to ensure accurate temporal alignment of physiological data.

Various combinations of the above and below recited features, embodiments, implementations, and aspects are also disclosed and contemplated by the present disclosure.

Additional implementations of the disclosure are described below in reference to the appended claims, which may serve as an additional summary of the disclosure.

In various implementations, systems and/or computer systems are disclosed that comprise a computer-readable storage medium having program instructions embodied therewith, and one or more processors configured to execute the program instructions to cause the systems and/or computer systems to perform operations comprising one or more aspects of the above- and/or below-described implementations (including one or more aspects of the appended claims).

In various implementations, methods and/or computer-implemented methods are disclosed in which, by one or more processors executing program instructions, one or more aspects of the above- and/or below-described implementations (including one or more aspects of the appended claims) are implemented and/or performed.

In various implementations, computer program products comprising a computer-readable storage medium are disclosed, wherein the computer-readable storage medium has program instructions embodied therewith, the program instructions executable by one or more processors to cause the one or more processors to perform operations comprising one or more aspects of the above- and/or below-described implementations (including one or more aspects of the appended claims).

### BRIEF DESCRIPTION OF THE DRAWINGS

Various implementations will be described hereinafter with reference to the accompanying drawings. These implementations are illustrated and described by example only, and are not intended to limit the scope of the disclosure. In the drawings, similar elements may have similar reference numerals.

FIGS. 1A-1B are schematic block diagrams illustrating example implementations of a physiological monitoring system (PMS).

FIG. 1C illustrates an example implementation of a physiological monitoring system (PMS).

FIG. 1D is a schematic block diagram illustrating an implementation of an example physiological monitoring system (PMS).

FIG. 2 is a block diagram illustrating an example implementation of a monitoring hub of a physiological monitoring system (PMS).

FIGS. 3A-3B are perspective views of example monitoring hubs.

FIG. 4 is a flowchart illustrating an example process of generating patient alarms.

### DETAILED DESCRIPTION

Although certain implementations, embodiments, and examples are disclosed below, the inventive subject matter extends beyond the specifically disclosed implementations to other alternative implementations and/or uses and to modifications and equivalents thereof. Thus, the scope of the claims appended hereto is not limited by any of the particular implementations described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain implementations; however, the order of description should not be construed to imply that these operations are order dependent. Additionally, the structures, systems, and/or devices described herein may be embodied as integrated components or as separate components. For purposes of comparing various implementations, certain aspects and advantages of these implementations are described. Not necessarily all such aspects or advantages are achieved by any particular implementation. Thus, for example, various implementations may be carried out in a manner that achieves or optimizes one advantage or group of advantages as taught herein without necessarily achieving other aspects or advantages as may also be taught or suggested herein.

### Overview

A physiological monitoring system (PMS) can monitor a patient or subject including physiological data of the subject. One or more physiological sensors can be coupled to the subject and can obtain physiological data of the subject. The one or more sensors can communicate physiological data to a monitoring hub which can display indicia of the physiological data. The one or more sensors can communicate physiological data to one or more servers, such as via a monitoring hub. The one or more servers can process the physiological data to generate processed physiological data, which can include physiological parameter values, alarms, alerts, notifications, trends, comparisons, and/or the like. The server(s) can perform one or more operations, functions, or algorithms, such as data filtering, data averaging, identifying maximums or minimums, identifying trends, extrapolating data, interpolating data, and/or the like. The server(s) can continuously measure physiological data of a subject and/or monitor such physiological data in real-time.

The server(s) can aggregate and cross-pollinate physiological data originating from various different physiological sensors, monitors, and/or other distributed devices that are collecting and/or transmitting the physiological data. Cross-pollination of physiological data can include comparing, integrating (e.g., data fusion), analyzing, and/or or arbitrating between the aggregated physiological data obtained from the multiple different, distributed monitoring devices, such as to determine various relationships between different physiological parameters. For example, a reduction in oxygen saturation of a subject coupled with an increase in heart rate of the subject may be indicative of an adverse respiratory event. The server(s) may determine an overall medical characterization of a subject based on the cross-pollinated physiological data, including a wellness index and/or risk index, such as described in greater detail in at least U.S. patent application Ser. No. 13/371,767, filed on Feb. 13, 2012, entitled “MEDICAL CHARACTERIZATION SYSTEM,” and in at least non-patent publication entitled “Halo: Assessing Global Patient Status with the Halo Index,” the entire contents of both of which are hereby incorporated herein by reference.

The server(s) can generate one or more alarms and/or update one or more alarm threshold conditions associated with one or more physiological parameters, such as based on the cross-pollinated physiological data. For example, alarm threshold conditions and related generated alarms may be associated with the medical characterization of the subject, such as with the wellness or risk index of the subject. In some implementations, alarm threshold conditions can include dynamic and/or adaptive thresholds. For example, the server(s) may continuously update alarm threshold conditions in real-time as the server(s) continuously aggregate and cross-pollinate physiological data received from the various different, distributed physiological monitoring devices.

In some implementations, alarm threshold conditions determined and used by the server(s) for generating alarms may be different than those used by any of the distributed physiological monitoring devices, such as those used by a sensor and/or monitoring hub. For example, the server(s) may implement a complex alarm algorithm that takes in multiple different physiological inputs and triggers an alarm based on a combination of multiple different physiological parameters. The server(s), via the complex algorithm, may analyze data associated with multiple different physiological parameters simultaneously and determine patterns and/or trends based on various different combinations of data. The complex algorithm may also intake contextual inputs (e.g., age of a subject, medical history of a subject, presence of certain physiological conditions) to further refine alarm threshold conditions. Distributed physiological monitoring devices (e.g., sensors, monitoring hubs), however, may implement a simple alarm algorithm, such as a simple physiological threshold alarm as further described herein. The sensors and/or monitoring hubs may generate alarms using the simple alarm algorithm when connection between the sensors and/or monitoring hubs and the server(s) is interrupted or lost. In some implementations, the sensors and/or monitoring hubs may generate alarms using the simple alarm algorithm only when connection to the server(s) is interrupted or lost.

The server(s) can cause generated alarm(s) to be output by one or more distributed devices via, for example, alarm signals. The server(s) may cause output of the alarm(s) such as in a display of a distributed device, by a speaker of a distributed device, by other distributed displays and/or loudspeakers, by a vibration of a distributed device, and/or the like.

In some cases, communication connection between the server(s) and one or more distributed devices (e.g., sensors, monitors, etc.) can be interrupted or lost. In such instances, the one or more distributed devices may take over the physiological monitoring of a subject and the associated alarming. Described herein are systems, devices, processes, etc., for transferring physiological monitoring and associated alarming from the server(s) to the distribute devices in instances of server communication connection failure (e.g., when connection between one or more distributed devices and one or more servers is interrupted or lost), which can provide numerous benefits including improved physiological monitoring, improved health care services, and the like.

Advantageously, the systems, devices, and processes described herein can facilitate continued (e.g., uninterrupted) monitoring of a subject's physiological condition. For example, when connection to one or more servers is interrupted or lost, one or more distributed devices may continue physiological monitoring of the subject, such as at the local level. The distributed device(s) may take over physiological monitoring of a subject without interruption to the collection of physiological data. The distributed device(s) can continuously process a subject's physiological data in real-time until connection to the server(s) is improved or reestablished. The distribute devices can generate alarms based on the processed data, such as alarms relating to adverse events. The distributed devices may output alarms to notify medical practitioners of an adverse event. This can help ensure that, in the event of server connection failure, medical practitioners continue to receive important physiological information associated with patients, and that patients continue to receive care from medical practitioners. Maintaining uninterrupted, continuous, and real-time monitoring can be critical for timely medical intervention, such as in the case of an adverse event, especially when medical practitioners may otherwise by unaware of a server connection failure.

Advantageously, the systems, devices, and processes described herein can prevent loss of physiological data. For example, the server(s) can be configured to maintain a data buffer of physiological sensor data. The server(s) can maintain (e.g., not delete, discard, etc.) the buffer when physiological data is not received from one or more distributed devices, such as during a server connection failure. The server(s) can extrapolate and/or interpolate data into the buffer at times corresponding to when physiological data is not being received from one or more distributed devices so as to increase reliable data points on which further processing of the sensor data may be based (e.g., determining and outputting final physiological parameter values, determining alarm conditions, generating alarms, etc.). The server(s) may generate alarms based on data stored in the buffer (e.g., based on interpolated and/or extrapolated data). Advantageously, the server(s) can preserve physiologically accurate measurements even during server connection failures when physiological data is not being received from physiological sensors and/or monitoring hubs and generate alarms accordingly. Upon reconnection, with one or more distributed devices, the server(s) may send alarm signals (associated with alarms based on the stored data) to such devices for output of an alarm. In a similar manner, distributed device(s) may store physiological data in local memory (such as in a buffer) until connection to one or more servers is reestablished. The distributed device(s) may automatically transmit the stored physiological data (e.g., unprocessed or processed data) to the server(s) for further processing upon reestablishing connection. Advantageously, such transitions between device and server can help reduce (or minimize) any delay in detecting adverse event and/or attending to adverse events by a medical practitioner, such as when servers and distributed devices are reestablishing connection and transfer of physiological data between each other.

To facilitate an understanding of the systems and methods discussed herein, several terms are described below. These terms, as well as other terms used herein, should be construed to include the provided descriptions, the ordinary and customary meanings of the terms, and/or any other implied meaning for the respective terms, wherein such construction is consistent with context of the term. Thus, the descriptions below do not limit the meaning of these terms, but only provide example descriptions.

In some implementations, a sensor can comprise a wearable device. A sensor can comprise a wireless wearable device. A wearable device can include one or more sensors. A wearable device can comprise a wearable hub in communication with one or more sensors. A sensor can comprise an auricular device such as an earbud, earpiece, headphone, earphone, or the like. A sensor can comprise a wrist-worn device such as a smartwatch. A sensor can comprise a physiological sensor. A sensor can collect physiological data from a subject such as ECG data, EEG data, blood oxygenation data, heart rate data, pulse data, respiration data, blood pressure data, motion data, orientation data, temperature data, etc. A sensor can be coupled to a subject. A sensor may be attached to a subject. A sensor can be donned by a subject. A sensor can be worn by a subject. A sensor can be secured to a subject by adhesion. A sensor can be secured to a subject by one or more straps. A sensor may be worn on and/or attached to a finger, wrist, arm, forearm, head, forehead, ear, chest, back, torso, stomach, leg, ankle, foot, toe, or other body portion of a subject. A plurality of sensors can be disposed within a same housing or device. Sensors may be disposed within separate housings or devices.

In some implementations, a monitoring hub can comprise an electronic device configured to facilitate physiological monitoring of a patient. A monitoring hub can display indicia corresponding to physiological data of the patient. A monitoring hub can be mobile. A monitoring hub can be portable. A monitoring hub may comprise a hand-held device. A monitoring hub may be carried by a user. A monitoring hub can be mounted to a wall. A monitoring hub may be an in-room display. A monitoring hub may be stationary. A monitoring hub may be at a fixed location. A monitoring hub may comprise a display, tablet, monitor, PC, phone, laptop, wearable device, such as a smartwatch, or the like. A monitoring hub may communicate with one or more remote computing devices via one or more wireless communication protocols. A monitoring hub may communicate with a remote server. A monitoring hub may communicate with one or more sensors. A monitoring hub may also be referred to herein as a hub, an electronic device, a display device, a display terminal, a monitoring device, or the like. An environment, such as a healthcare facility may have a plurality of monitoring hubs distributed throughout the environment at various locations such as mounted to walls, mounted to doors, distributed in rooms, distributed along hallways, carried by personnel, coupled to beds, coupled to patients, or the like.

In some implementations, an origin monitoring hub can refer to a monitoring hub in wireless communication with one or more sensors prior to transferring physiological monitoring to another monitoring hub. An origin monitoring hub can comprise any of the example monitoring hubs shown and/or described herein including structural and/or operational features of any of the example monitoring hubs shown and/or described herein. An origin monitoring hub may also be referred to herein as a first monitoring hub, an initial monitoring hub, or the like.

In some implementations, a destination monitoring hub can refer to a monitoring hub in wireless communication with one or more sensors subsequent to transferring physiological monitoring from another monitoring hub. A destination monitoring hub can comprise any of the example monitoring hubs shown and/or described herein including structural and/or operational features of any of the example monitoring hubs shown and/or described herein. A destination monitoring hub may also be referred to herein as a second monitoring hub, a subsequent monitoring hub, another monitoring hub, or the like.

A destination monitoring hub may comprise a different type of monitoring hub than an origin monitoring hub. For example, one of the destination monitoring hub or the origin monitoring hub may comprise a mobile monitoring hub while the other of the destination monitoring hub or the origin monitoring hub comprises a monitoring hub in a fixed location, such as a wall-mounted monitoring hub or an in-room monitoring hub. A destination monitoring hub may comprise a same type of monitoring hub as an origin monitoring hub. For example, a destination monitoring hub and an origin monitoring hub may both comprise mobile monitoring hubs.

In some implementations, “transferring physiological monitoring” can refer to transferring the monitoring, displaying, and/or collection of physiological data from an origin monitoring hub to a destination monitoring hub. Transferring physiological monitoring can include transferring a wireless connection between sensor(s) and an origin monitoring hub to sensor(s) and a destination monitoring hub. Transferring physiological monitoring can include updating or changing a wireless connection of physiological sensor(s) and/or updating or changing a wireless connection of monitoring hub(s). Transferring physiological monitoring can include terminating wireless communication between an origin monitoring hub and sensors. Transferring physiological monitoring can include establishing a wireless communication between a destination monitoring hub and sensors. In some implementations, transferring physiological monitoring can include transferring less than all of the wireless connections to sensors from an origin monitoring hub to a destination monitoring hub. In some implementations, transferring physiological monitoring can include transferring all of the wireless connections to sensors from an origin monitoring hub to a destination monitoring hub.

In some implementations, a transfer request can include a request to transfer physiological monitoring from an origin monitoring hub to a destination monitoring hub. A transfer request can be received via a user input at a monitoring hub. For example, a user may press a button on one or more monitoring hubs to initiate a transfer request. In some implementations, a transfer request may comprise a non-contact or minimal-contact user input, such as a wireless communication signal, facial recognition, eye recognition, fingerprint recognition, gesture recognition, voice recognition, or the like. A transfer request can be received at a location and/or computing device that is remote to a monitoring hub. A transfer request may initiate transferring physiological monitoring.

In some implementations, identification data can include data generated and/or received via a monitoring hub when transferring physiological monitoring. Identification data can include data that is associated with a user and can be used to identify the user. Identification data can comprise a user ID. Identification data can include a tag, marker, serial number, bar code, QR code, facial recognition, fingerprint recognition, voice recognition, eye recognition, gesture recognition, or the like. Identification data may be unique to a user. Identification data may be unique to a group of users (and may be the same for individuals within the group). Identification data may be used to identify a group to which the user belongs. Identification data may comprise or indicate permissions associated with a user such as permission or authority to transfer physiological monitoring. Identification data can include a reason (e.g., provided by a requesting user) for requesting a transfer. A computing device, such as a monitoring hub, can receive identification data via one or more wireless communication protocols such as near field communication (NFC) or radio frequency identification (RFID). For example, a user may place a badge configured for wireless communication in proximity to a monitoring hub to be detected by the monitoring hub. A computing device, such as a monitoring hub, can receive identification data via manual user input at a monitoring hub. For example, a user may enter their identification data at the monitoring hub via a keyboard, user interface, touchscreen, or the like. A computing device, such as a monitoring hub, can receive identification data via one or more biological markers. For example, a user may scan their finger, eye, face, or speak as their identification data to be identified at the monitoring hub. Identification data can be linked or paired with a transfer request.

In some implementations, a transfer request status may indicate a status of a request to transfer physiological monitoring. A transfer request status can include an approved or not approved status. In some implementations, a transfer request may be approved if identification data from a first monitoring hub matches identification data from a second monitoring hub. A transfer request may be approved if a requesting user has appropriate permissions to perform the transfer. In some implementations, a transfer request may not be approved if identification data from a first monitoring hub does not match identification data from a second monitoring hub. A transfer request may not be approved if a requesting user does not have appropriate permissions to perform the transfer.

In some implementations, wireless communication configuration data can comprise data used to establish wireless communication between one or more computing devices. For example, a monitoring hub and sensor may implement wireless communication configuration data to communicate with each other via one or more wireless communication protocols. Wireless communication configuration data can include device addresses of one or more computing devices such as monitoring hubs and/or sensors. Wireless communication configuration data can include access codes such as one or more of Inquiry Access Codes (IAC), Device Access Codes (DAC), and Channel Access Codes (CAC). An access code can include and/or be derived from a device address. Wireless communication configuration data can include link keys. Wireless communication configuration data can include clock data such as frequencies at which computing devices will communicate (e.g., to transmit data). Wireless communication configuration data may also be referred to herein as wireless communication data or communication data or wireless configuration data or configuration data.

In some implementations, a device address can facilitate wireless communication between computing devices. A device address may be associated with a computing device. A device address may be unique to a computing device. A device address can comprise an IP address. A device address can comprise a MAC address. A device address can comprise a serial ID associated with a computing device. A device address can comprise a Bluetooth Address (BD_ADDR). A device address can comprise an LAP value. A device address, or derivation thereof, may form at least a portion of an access code.

In some implementations, a link key may facilitate wireless communication between computing devices. A link key can authenticate one or more computing devices with each other. A link key can encrypt data exchanged wirelessly between one or more computing devices. A link key can comprise a Long-Term Key (LTK).

In some implementations, physiological data can include data generated by one or more sensors. Physiological data can correspond to a subject. Physiological data can include raw data, partially processed data, and/or fully processed data. Physiological data can include physiological parameters. Physiological data can include data relating to heart rate, respiration rate, blood pressure, blood oxygen saturation, hemoglobin content, PPG data, ECG data, EEG data, temperature, subject orientation, subject position, subject movement, depth-of-consciousness, capnography data, acoustic data, motion data, as non-limiting examples. Physiological data can include historical physiological data. Historical physiological data can include physiological data generated by a sensor over a time frame preceding a present time. Historical physiological data can include data corresponding to a time frame of less than 24 hours, less than 12 hours, less than 1 hour, less than 30 minutes, less than 10 minutes, less than 5 minutes, less than 2 minutes, less than 1 minute, less than 30 seconds, less than 15 seconds, less than 10 seconds, less than 5 seconds, or less than 1 second. Physiological data can include real-time physiological data. Real-time physiological data can include physiological data transmitted and/or received at a substantially similar time as the physiological data is generated by a sensor, for example, such that any difference in time may be imperceptible to human senses.

Various implementations of the systems and methods disclosed herein may be utilized for continuous measurement of a subject's physiological data and/or to monitor such physiological data in real time. Continuous monitoring can include obtaining physiological parameter data (e.g., one or more signals indicative of a physiological parameter) from one or more sensors (such as any of the sensors disclosed herein) at a sampling rate sufficient to consistently capture irregular physiological events while the user is in contact with the wearable device. Continuous monitoring may include sampling physiological data at a sampling rate until the patient monitoring system (e.g., a wearable sensor, a monitoring hub, server(s), etc.) identifies a likeliness of a physiological event and then may adjust the sampling rate (e.g., increase or decrease the sampling rate) to better capture physiological parameter data regarding the event. Capturing such changes may be useful for identifying a physiological event or other health condition. Such continuous measurement may allow for monitoring physiological trends and/or for generating a smoother waveform based on the physiological data, which may provide additional information in regard to corresponding physiological parameters. A sampling rate used for continuous measurement may be determined, in some examples, based on a rate at which meaningful changes in physiological data are reasonably anticipated. In some examples, a sampling rate can be defined by relatively short time periods such that even minor changes in the physiological data over time are captured. For example, continuous measurement may include obtaining physiological parameter data at a sampling rate of 1 minute, 30 seconds, 24 seconds, 12 seconds, 11 seconds, 10 seconds, 9 seconds, 8 seconds, 7 seconds, 6 seconds, 5 seconds, 4 seconds, 3 seconds, 2 seconds, 1 second, 0.5 seconds, 0.2 seconds, 0.1 seconds, 0.05 seconds, 0.04 seconds, 0.02 seconds, 0.01 seconds, 0.005 seconds, 0.004 seconds, 0.002 seconds, 0.001 seconds, 0.0005 seconds, 0.00025 seconds, 0.0002 seconds, 0.000125 seconds, 0.0001 seconds, or rates therebetween. Faster rates are also possible. A continuous sampling rate may, in some examples, be selected or updated based on anticipated changes in monitored physiological data, sensor type, physiological parameter being monitored or tracked, hardware considerations, or other physiological or hardware considerations.

Monitoring physiological data in real-time may include processing, transmitting, and/or displaying such physiological data within a short time period after such data is obtained, for example, within 10 seconds, within 5 seconds, within 4 seconds, within 3 seconds, within 2 seconds, within 1 second, within 0.9 seconds, within 0.7 seconds, within 0.5 seconds, within 0.3 seconds, or within 0.1 seconds from when such data is obtained. Real-time monitoring, especially when paired with continuous measurement, can facilitate improved monitoring and treatment, if needed, of physiological conditions that may change in relatively short time periods. This can in turn facilitate accurate detection of changes in physiological parameters and rapid response to such changes (if necessary).

Continuous monitoring of physiological parameters by a PMS can be beneficial to the wearer. Continuous monitoring can advantageously improve detection of critical events such as oxygen desaturation events, heart palpitations, or the like. Continuous monitoring can advantageously allow for early detection of physiological abnormalities, such as cardiovascular abnormalities (e.g., arrhythmias, etc.), respiratory abnormalities (e.g., hypoxia, sleep apnea, etc.), or the like. In some examples, health conditions can be monitored while the user is asleep. Continuous monitoring can enable timely medical intervention, which can be crucial in preventing complications or worsening of a physiological condition. Continuous monitoring can lead to improved management of chronic diseases by, for example, keeping track of changes in physiological parameters so that wearers of the wearable device can be proactive in their healthcare routines. Advantageously, continuous monitoring can help to tailor healthcare solutions to individual needs based on, for example, personal physiological data trends.

### Example Systems

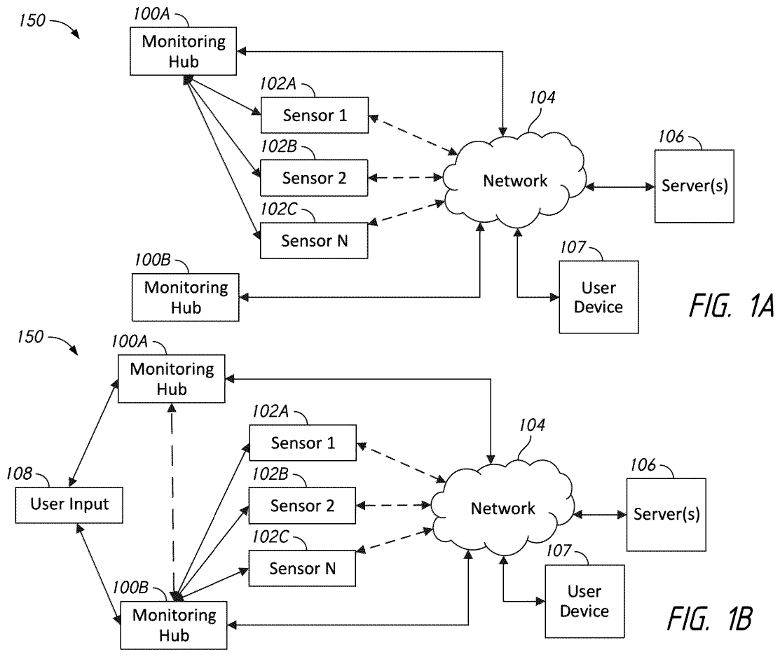

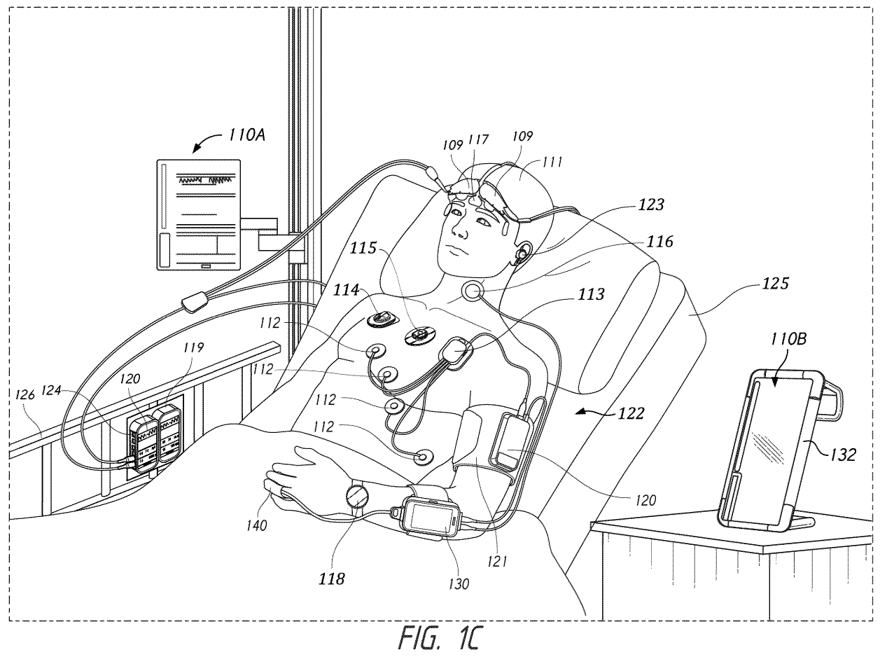

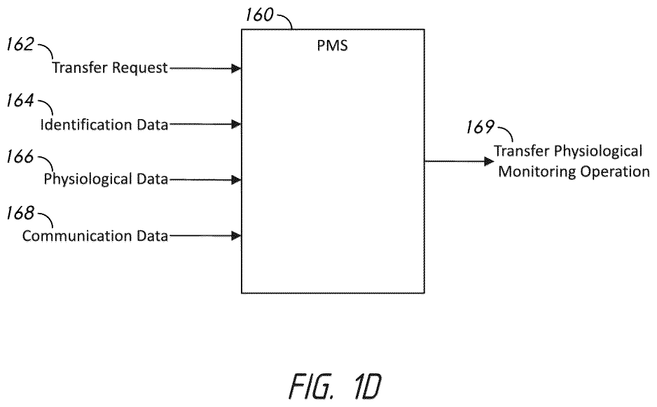

FIG. 1A is a schematic block diagram illustrating an example implementation of a physiological monitoring system (PMS) 150. The PMS 150 can include a monitoring hub 100A, a monitoring hub 100B, one or more sensors 102 (e.g., sensors 102A, 102B, 102C), a network 104, a user device 107, and one or more servers 106. In some implementations, the PMS 150 may include only two monitoring hubs (e.g., hubs 100A, 100B). In some implementations, the PMS 150 may include more than two monitoring hubs. In some implementations, the PMS 150 may include only one monitoring hub.

The monitoring hub 100A can communicate with the one or more sensors 102. In some implementations, the monitoring hub 100A may be in wired (or wire-like) communication with one or more sensors 102 via a wire or cable connection or any other suitable electronic connection that can permit transfer of data between the monitoring hub 100A and the one or more sensors 102. In some implementations, the monitoring hub 100A may be in wireless connection with the one or more sensors 102 via any variety of communication protocols, including near-field communication protocols and far-field communication protocols. Near-field communication (NFC) protocols, which may also be referred to as non-radiative communication, can implement inductive coupling between coils of wire to transfer energy via magnetic fields (e.g., NFMI). Near-field communication protocols can implement capacitive coupling between conductive electrodes to transfer energy via electric fields. Far-field communication protocols, which may also be referred to as radiative communication, can transfer energy via electromagnetic radiation (e.g., radio waves). In some implementations the monitoring hub 100A may communicate with the one or more sensors 102 via any variety of communication protocols such as Wi-Fi (e.g., 2.4 GHz channel, 5 GHz channel), Bluetooth® (e.g., Bluetooth Low Energy 5.0/Mesh), ZigBee®, Z-wave®, cellular telephony, 1G, 2G, 3G, 4G, 5G, infrared, near-field communication (NFC), radio frequency identification (RFID), satellite transmission, inductive coupling, capacitive coupling, proprietary protocols, any combination of the foregoing, and/or any other suitable wireless connection. In some implementations, the sensor(s) 102 may be a “slave” in a master-slave communication relationship such as a Bluetooth communication protocol with the monitoring hub 100A. In some implementations, the sensor(s) 102 may communicate with only one device at a time (e.g., a “master” device) such as a monitoring hub. The monitoring hub 100A may communicate data to the one or more sensors 102 and/or receive data from the one or more sensors 102. For example, the monitoring hub 100A may receive physiological data from the one or more sensors 102. In some examples, the monitoring hub 100A may receive communication data (e.g., device addresses of the one or more sensors 102) from the one or more sensors 102 and/or communicate communication data (e.g., device addresses of the monitoring hubs 100A, 100B) to the one or more sensors 102. In the example implementation illustrated in FIG. 1A, monitoring hub 100B has not established direct wireless communication with the one or more sensors 102.

The monitoring hubs 100A, 100B can communicate with the server(s) 106 via a network 104. The network 104 can include any one or more communications networks. The network 104 can include a plurality of computing devices configured to communicate with one another. The network 104 can include routers. The network 104 can include the Internet. The network 104 can include any combination of networks, such as a personal area network (PAN), a local area network (LAN), a metropolitan area network (MAN), a wide area network (WAN), or the like. Accordingly, various components of the PMS 150 can communicate with one another directly or indirectly via any appropriate communications links and/or networks, such as network 104 (e.g., one or more communications links, one or more computer networks, one or more wired or wireless connections, the Internet, any combination of the foregoing, and/or the like). The monitoring hubs 100A, 100B may, via the network 104, communicate data to the server(s) 106 and/or receive data from the server(s) 106 including communication data (e.g., device addresses and/or link keys corresponding to the sensors 102 and/or monitoring hubs 100), physiological data, identification data (user ID), transfer requests, request approval status, or the like. The monitoring hubs 100A, 100B may communicate with the server(s) 106 via any variety of communication protocols such as Wi-Fi (e.g., 2.4 GHZ channel, 5 GHz channel), Bluetooth® (e.g., Bluetooth Low Energy 5.0/Mesh), ZigBee®, Z-wave®, cellular telephony, 1G, 2G, 3G, 4G, 5G, infrared, near-field communication (NFC), radio frequency identification (RFID), satellite transmission, inductive coupling, capacitive coupling, proprietary protocols, any combination of the foregoing, and/or any other suitable wireless connection. In some implementations, a monitoring hub 100 may communicate with the server(s) 106 via a different wireless communication protocol than which it communicates with the one or more sensors 102. For example, a monitoring hub 100 may communicate with the server(s) 106 via a first wireless communication protocol, such as Wi-Fi, and may communicate with the one or more sensors 102 via a second wireless communication protocol, such as Bluetooth.

In some implementations, the network 104 can be configured to handle increased data traffic as sensors, monitoring hubs, servers, and/or other computing and/or physiological monitoring devices are added to PMS 150. For example, the network 104 can include hardware such as switches, routers, WAN optimization devices, other connection points and the like for handling increased data throughput and more connections simultaneously. In some examples, the network 104 may implement virtual local area networks (“VLANs”) to segment traffic within specific areas of the network. In some examples, the network 104 can include quality-of-service rules to set priorities for certain data traffic. In some aspects, the network 104 may implement advanced routing techniques to adapt network conditions to find efficient paths for data traffic. For example, the network 104 can implement dynamic routing, load balancing, and the like.

In some implementations, the sensors 102 may optionally communicate with the server(s) 106 via the network 104. For example, the sensors 102 may communicate physiological data to the server(s) 106 and/or receive communication data (e.g., device address of a monitoring hub) from the server(s) 106. In some implementations, the sensors 102 may not communicate directly with the server(s) 106. In some implementations, data may be transmitted from the sensor(s) 102 to the server(s) 106 via a monitoring hub 100, or vice versa.

In some implementations, the monitoring hub 100A may be portable or mobile. For example, the monitoring hub 100A may be sized, shaped, and/or include a housing or casing to facilitate carrying the monitoring hub 100A such as by hand. In some implementations, the monitoring hub 100A may be stationary or fixed in a location. For example, the monitoring hub 100A may be mounted to a wall. In some implementations, the monitoring hub 100B may include similar structural and/or operational features as monitoring hub 100A. The monitoring hub 100A may be referred to as an origin monitoring hub. The monitoring hub 100B may be referred to herein as a destination monitoring hub. In some implementations, the monitoring hub 100A and/or the monitoring hub 100B can manage and/or process data received from the one or more sensors 102. For example, the monitoring hub 100A and/or the monitoring hub 100B can process physiological data originating at sensors 102 to generate processed physiological data which can include physiological parameter values, alarms, alerts, notifications, trends, comparisons, and/or the like. In some examples, the monitoring hub 100A and/or the monitoring hub 100B can be configured to perform one or more operations, functions, or algorithms, such as data filtering, data averaging, identifying maximums or minimums, identifying trends, extrapolating data, interpolating data, and/or the like. The monitoring hub 100A and/or the monitoring hub 100B can continuously measure physiological data of a subject and/or monitor such physiological data in real-time. In this manner, the monitoring hubs 100A, 100B can include hardware capabilities for continuous and/or real-time processing.

Monitoring hub 100A and/or monitoring hub 100B can manage and/or process data received from multiple different, distributed physiological sensors. The monitoring hubs 100A and/or 100B can aggregate and cross-pollinate the physiological data originating from the multiple different physiological sensors that are collecting and/or transmitting the physiological data. The monitoring hub 100A and/or the monitoring hub 100B can compare, integrate (e.g., data fusion), analyze and/or arbitrate between the aggregated physiological data obtained from the multiple different physiological sensors, such as to determine various relationships between different physiological parameters. For example, a determined reduction in oxygen saturation of a subject coupled with a determined increase in heart rate of the subject may be indicative of an adverse respiratory event. The monitoring hubs 100A and/or 100B may determine an overall medical characterization of a subject based on the cross-pollinated physiological data, such as a wellness index and/or risk index. Determination of an overall wellness or risk index is described in greater detail in at least U.S. patent application Ser. No. 13/371,767 and in at least non-patent publication entitled “Halo: Assessing Global Patient Status with the Halo Index.”The monitoring hub 100A and/or monitoring hub 100B can generate one or more alarms and/or update one or more alarm threshold conditions associated with one or more physiological parameters, such as based on the cross-pollinated physiological data. For example, alarm threshold conditions and related generated alarms may be associated with the medical characterization of the subject, such as with the wellness or risk index of the subject. The monitoring hubs 100A and/or 100B may continuously update alarm threshold conditions in real-time as the monitoring hubs 100A and/or 100B continuously aggregate and cross-pollinate physiological data received from the multiple different, distributed physiological sensors. Patient monitors capable of measuring pulse oximetry parameters, such as SpO2 and pulse rate in addition to advanced parameters, such as HbCO, HbMet and total hemoglobin (Hbt, THb, or SpHb) and corresponding multiple wavelength optical sensors are described in at least U.S. patent application Ser. No. 11/367,013, filed Mar. 1, 2006, entitled “MULTIPLE WAVELENGTH SENSOR EMITTERS” and U.S. patent application Ser. No. 11/366,208, filed Mar. 1, 2006, entitled “NONINVASIVE MULTI-PARAMETER PATIENT MONITOR,” the entire contents of both of which are hereby incorporated by reference herein. Further, noninvasive blood parameter monitors and corresponding multiple wavelength optical sensors, such as Rainbow™ adhesive and reusable sensors and RAD-57™ and Radical-7™ monitors for measuring SpO2, pulse rate, perfusion index, signal quality, HbCO, and HbMet among other parameters are available from Masimo Corporation, Irvine, CA (Masimo).

The one or more sensors 102 can include various types of sensors configured to collect physiological data of a subject. The one or more sensors 102 can attach or couple to different parts of a subject such as, but not limited to, arms, legs, torso, chest, head, neck, fingers, forehead, and the like. The one or more sensors 102 can collect patient physiological data including, but not limited to, data relating to heart rate, pulse rate, respiration rate, blood pressure, blood oxygen saturation, hemoglobin content, ECG data, EEG data, temperature, subject orientation, subject position, subject movement, as non-limiting examples, and the like. The one or more sensors 102 can transmit physiological data to the monitoring hub 100A, monitoring hub 100B, and/or to the server(s) 106 in real-time as the one or more sensors 102 collect the data. In some implementations, one or more sensors 102 can include processors that can fully or partially process the data obtained by the sensors 102. For example, the one or more sensors 102 can process physiological data obtained by the sensors 102 to generate processed physiological data which can include physiological parameter values, alarms, alerts, notifications, trends, comparisons, and/or the like. In some examples, the one or more sensors 102 can be configured to perform one or more operations, functions, or algorithms, such as data filtering, data averaging, identifying maximums or minimums, identifying trends, extrapolating data, interpolating data, and/or the like. In some implementations, one or more sensors 102 can continuously measure physiological data of a subject and/or monitor such physiological data in real-time. In this manner, the one or more sensors 102 can include hardware capabilities for continuous and/or real-time processing. Patient monitors capable of measuring pulse oximetry parameters, such as SpO2 and pulse rate in addition to advanced parameters, such as HbCO, HbMet and total hemoglobin (Hbt, THb, or SpHb) and corresponding multiple wavelength optical sensors are described in at least U.S. patent application Ser. No. 11/367,013 and U.S. patent application Ser. No. 11/366,208.

The server(s) 106 may comprise one or more computing devices including one or more hardware processors. The one or more hardware processors may be configured to analyze, cleanse, edit, reduce, wrangle, or otherwise process data. The server(s) 106 may comprise program instructions configured to cause the server(s) 106 to perform one or more operations when executed by the hardware processors. The server(s) 106 may include, and/or have access to (e.g., be in communication with) a database or storage component or storage system which can include any computer readable storage medium and/or device (or collection of data storage mediums and/or devices), including, but not limited to, one or more memory devices that store data, including without limitation, dynamic and/or static random access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. In some implementations, the server(s) 106 may host a database which can be any data structure (and/or combinations of multiple data structures) for storing and/or organizing data, including, but not limited to, relational databases (e.g., Oracle databases, PostgreSQL databases, MySQL databases and the like), non-relational databases (e.g., NoSQL databases, and the like), in-memory databases, spreadsheets, as comma separated values (“CSV”) files, extensible markup language (“XML”) files, TeXT (“TXT”) files, flat files, spreadsheet files, and/or any other widely used or proprietary format for data storage. Databases can be stored in one or more data stores. In some implementations, the server(s) 106 may include and/or be in communication with a hosted storage environment that includes a collection of physical data storage devices that may be remotely accessible and may be rapidly provisioned as needed (commonly referred to as “cloud” storage).

Data stored in and/or accessible by the server(s) 106 can include processed and/or unprocessed physiological data including historical physiological data previously obtained by the one or more sensors 102 and/or communication data including, for example, link keys and/or device addresses associated with monitoring hubs, sensors, or the like. Data stored in and/or accessible by the server(s) 106 can include patient information such as a patient's name, date of birth, other personal information, billing and insurance information, unique identification number, date of admission to a healthcare facility, consent and authorization forms, assigned healthcare provider's name, assigned healthcare provider's credentials, assigned bed number, assigned room number, healthcare history, current health status, clinical notes, lab/diagnostic tests and/or results, imaging results, historical or planned procedures and/or interventions, allergies, treatment plans, prescribed medications, designated operating room, location, record of patient interactions (e.g., interactions with staff, visitors, and/or other patients), and/or other relevant patient-specific information. Data stored in and/or accessible by the server(s) 106 can also include healthcare practitioner information such as a healthcare practitioner's name, credentials, contact details, specialty, affiliations, and/or other relevant practitioner-specific information.

In some implementations, the network 104 may comprise and/or be in communication with an electronic medical records (EMR). In some implementations, the server(s) 106 may comprise and/or be in communication with an EMR. In some implementations, the one or more of the monitoring hubs 100A, 100B may be in communication with an EMR. An EMR can comprise a propriety EMR. An EMR can comprise an EMR associated with a hospital. An EMR can store data including medical records.

In some implementations, the server(s) 106 can manage and/or process data received from monitoring hub 100A and/or 100B. For example, the server(s) 106 can process physiological data originating at sensors 102 and received from monitoring hub 100A and/or 100B to generate processed physiological data which can include physiological parameter values, alarms, alerts, notifications, trends, comparisons, and/or the like. In some examples, the server(s) 106 can be configured to perform one or more operations, functions, or algorithms, such as data filtering, data averaging, identifying maximums or minimums, identifying trends, extrapolating data, interpolating data, and/or the like. The server(s) 106 can continuously measure physiological data of a subject and/or monitor such physiological data in real-time. In this manner, the server(s) 106 can include hardware capabilities for continuous and/or real-time processing.

In some implementations, the server(s) 106 may be configured to generate one or more alarms based at least in part on a determination of whether a physiological parameter satisfies an alarm condition, such as a physiological threshold condition. The server(s) 106 may be configured to generate one or more alarms according to multiple different physiological threshold conditions. For example, a threshold condition may be associated with each physiological parameter measurable by the server(s) 106. The physiological threshold condition can include limits such as lower and upper limits. In some implementations, the server(s) 106 can determine a change in the physiological data between a first time and a second time. For example, the server(s) 106 may combine the physiological data with physiological parameter time instances and determine a difference between beginning and ending timestamps associated with the physiological data. In some implementations, the server(s) 106 can determine whether the physiological data satisfies a physiological threshold condition and/or whether the physiological data satisfies a physiological threshold condition more than a certain number of times. In some implementations, the server(s) 106 can determine whether a value associated with the physiological data satisfies a physiological threshold condition. In some implementations, the server(s) 106 can determine whether a change in the physiological data satisfies a physiological threshold condition. In some implementations, the server(s) 106 can determine whether a rate of change of the physiological data satisfies a physiological threshold condition. In some implementations, the server(s) 106 can determine whether a change in the rate of change of the physiological data satisfies a physiological threshold condition. In some implementations, the server(s) 106 can determine whether the physiological data is changing in a certain direction (e.g., positive change or negative change). In some implementations, the server(s) 106 may make a determination based on any combination of the foregoing examples.

The physiological threshold condition(s) may be fixed or may be programmable. In some implementations, the physiological threshold condition(s) can be set and/or modified by a user utilizing a computing device (e.g., via a smartphone application, a mobile application, and/or a web application executing in a browser) in communication with the server(s) 106.

In some implementations, the physiological threshold conditions(s) may be based at least in part on a history of sensor data such as relating to an average of historical sensor data. In some implementations, the physiological threshold condition(s) may be based at least in part on aggregated and cross-pollinated physiological data, collected by server(s) 106 over time, from one or more sensors 102, monitoring hub 100A, monitoring hub 100B, other servers, and/or other physiological sensors and/or monitoring devices. The server(s) 106 may determine the physiological threshold condition(s) based on the plurality of physiological data, such as a plurality of physiological inputs originating from the one or more sensors 102, monitoring hub 100A, monitoring hub 100B, and/or other physiological sensors and/or monitoring devices. In some implementations, the physiological threshold condition(s) may be based on combinations of physiological data, such as data associated with multiple different physiological parameters and analyzed simultaneously by server(s) 106. In some implementations, the physiological threshold condition(s) can include dynamic and/or adaptive physiological threshold(s), such as based on determined patterns and/or trends associated with multiple different combinations of physiological inputs. In some implementations, the physiological threshold condition(s) can be based at least in part on contextual information (e.g., age of a subject, medical history of a subject, presence of certain physiological conditions) that the server(s) 106 can use to further refine and personalize the alarm threshold condition(s) to the subject.

In some implementations, the server(s) 106 may send to the one or more sensors 102, the monitoring hub 100A, and/or the monitoring hub 100B alarm configuration data indicative of the physiological threshold condition(s) to be used by the one or more sensors 102 and/or monitoring hubs 100A, 100B in determining whether to generate alarms. The alarm configuration data may be indicative of the physiological threshold condition(s) to be used by the one or more sensors 102 and/or monitoring hubs 100A, 100B to generate alarms in the event that communication connection between the server(s) 106 and the one or more sensors 102 and/or monitoring hubs 100A, 100B is interrupted or lost. For example, alarm configuration data may specify a physiological threshold condition to be used by the sensors 102 and/or monitoring hubs 100A, 100B. In some examples, the alarm configuration data may indicate a simple physiological threshold to be used by the sensors 102 and/or monitoring hubs 100A, 100B, as further described herein.

In some implementations, the server(s) 106 may send alarm signals, to the one or more sensors 102 and/or monitoring hubs 100A, 100B, indicative of alarming. For example, the server(s) 106 may generate an alarm based on a determination that a physiological parameter, trend, or other physiological data or patient information satisfies a physiological threshold condition. Additionally and/or alternatively, the alarm condition may take into account other measured information, such as patient measurement history, patient comorbidity indications, measurements from other parameters, types of procedures occurring and/or treatments administered or indicated, and/or other risk factors.

A physiological threshold condition may include, for example, where one or more physiological parameters, trends, or other physiological data exceeds a threshold condition, drops below a threshold condition, is outside of a threshold range, the like or a combination thereof. In some implementations, the physiological threshold condition may involve a plurality of physiological threshold conditions. The alarm condition may be pre-determined, and/or set in real time. The alarm condition may be dynamic, and/or adaptive, reflecting a combination of real-time and historical data, patient-specific characteristics, and clinical context, rather than relying solely on a fixed threshold for a single parameter.

The server(s) 106 may send alarm signals to the one or more sensors 102 and/or the monitoring hubs 100A, 100B such that the one or more sensors 102 and/or monitoring hubs 100A, 100B output the alarm. Outputting the alarm can include generating visual, auditory, and/or haptic alarms, such as by the one or more sensors 102 and/or the monitoring hubs 100A, 100B. For example, alarm signals received from the server(s) 106 can cause display of visual indicia of the alarm in a display of the monitoring hubs 100A, 100B and/or a display connected to the one or more sensors 102 (e.g., a wearable device). In some examples, alarm signals received from the server(s) 106 can cause a speaker of the monitoring hubs 100A, 100B and/or a speaker connected to the one or more sensors 102 (e.g., wearable device) to output a sound. In some examples, alarm signals received from the server(s) 106 can cause the monitoring hubs 100A, 100B (or portions thereof) and/or the one or more sensors 102 (e.g., wearable device) to vibrate. In some implementations, the server(s) 106 may send alarm signals to other devices (e.g., other devices inside of or outside of a patient room), such as to a display terminal, a loudspeaker, and/or other alarm system. For example, the server(s) 106 may send alarm signals to an alarm system located in the ceiling of a patient room for output of the alarm, such as further described in U.S. patent application Ser. No. 18/123,131, filed on Mar. 17, 2023, entitled “MODULAR WIRELESS PHYSIOLOGICAL PARAMETER SYSTEM,” the entire contents of which are hereby incorporated herein by reference.

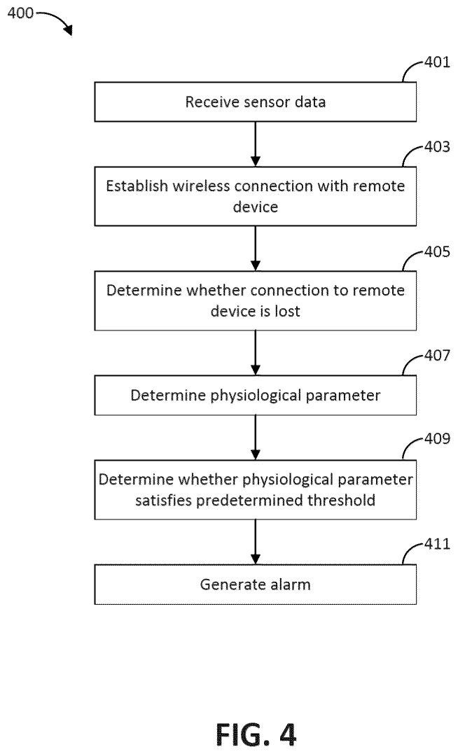

In some cases, communication connection between the server(s) 106 and the one or more sensors 102 and/or monitoring hubs 100A, 100B may be interrupted or lost. The one or more sensors 102 and/or the monitoring hubs 100A, 100B may determine that communication connection with the server(s) 106 is interrupted or lost, such as further described with reference to FIG. 4. In such instances, the one or more sensors 102, the monitoring hub 100A, and/or the monitoring hub 100B can process the physiological signals originating from the one or more sensors 102, such as until communication connection with the server(s) 106 is improved (e.g., connection strength increased) or reestablished. For example, the one or more sensors 102 and/or the monitoring hubs 100A, 100B can process the physiological signals originating at sensors 102 to generate processed physiological data, which can include physiological parameter values, alarms, alerts, notifications, trends, comparisons, and/or the like. In some examples, the server(s) 106 can be configured to perform one or more operations, functions, or algorithms, such as data filtering, data averaging, identifying maximums or minimums, identifying trends, extrapolating data, interpolating data, and/or the like.

In response to a determination that connection between the server(s) 106 and the one or more sensors 102 and/or monitoring hubs 100A, 100B is interrupted or lost, the sensors 102 and/or monitoring hubs 100A, 100B may be configured to generate and send out a connection alarm indicative of interrupted or lost connection with the server(s) 106. The connection alarm can include visual, auditory, and/or haptic alarm, such as visual, auditory, and/or haptic feedback. For example, a display of the sensors 102 and/or monitoring hubs 100A, 100B may render and display visual indicia indicative of interrupted or lost connection. In some examples, an audio device (e.g., speakers) of the sensors 102 and/or monitoring hubs 100A, 100B may output audio indicative of interrupted or lost connection. In some examples, the sensors 102 and/or monitoring hubs 100A, 100B can include status indicators (e.g., LEDs) configured to emit light when connection to server(s) 106 is interrupted or lost. In some examples, at least a portion of the sensors 102 and/or monitoring hubs 100A, 100B may vibrate in response to interrupted or lost connection with server(s) 106. In some implementations, the one or more sensors 102 and/or monitoring hubs 100A, 100B may send alarm signals to other distributed monitoring devices (e.g., display terminal, loudspeakers, etc.) to output alarms indicative of a server connection failure.

In some implementations, the one or more sensors 102 and/or the monitoring hubs 100A, 100B can determine whether a physiological parameter satisfies a physiological alarm condition, and generate an alarm in response to the determination. Normally, alarms are generated by the server(s) 106 using a relatively complex algorithm and not be the sensors 102 or monitoring hubs 100A, 100B. For example, the one or more sensors 102 and/or the monitoring hubs 100A, 100B may be configured to generate alarms only when connection to the server(s) 106 is interrupted or lost. In some implementations, the one or more sensors 102 and/or the monitoring hubs 100A, 100B may determine whether a value, a change, a rate of change, or a change in the rate of change of the physiological data satisfies a physiological alarm condition and/or satisfies a physiological alarm condition a certain number of times.

The physiological alarm condition used by the one or more sensors 102 and/or the monitoring hubs 100A, 100B for generating alarms may be implemented via a relatively simple alarm algorithm that includes for example, a predetermined physiological threshold. The predetermined threshold may be preloaded onto the one or more sensors 102 and/or the monitoring hubs 100A, 100B, and stored in local memory. In some implementations, the predetermined threshold can be based on alarm configuration data received from the server(s) 106, such as prior to interruption or loss of connection with the server(s) 106. In some implementations, the one or more sensors 102 and/or the monitoring hubs 100A, 100B may be configured to generate alarms (such as according to the predetermined threshold) only when communication connection with the server(s) 106 is interrupted or lost. Advantageously, transferring physiological data processing and alarming to the one or more sensors 102 and/or monitoring hubs 100A, 100B when connection to server(s) 106 is interrupted or lost can provide continued (e.g., uninterrupted) monitoring of a subject's (e.g., patient's) physiological condition. This can help ensure that, in the event of server connection failure, medical practitioners continue to receive important physiological information associated with patients, and that patients continue to receive care from medical practitioners. Maintaining uninterrupted, continuous, and real-time monitoring can be critical for timely medical intervention, such as in the case of an adverse event, especially when medical practitioners may otherwise be unaware of a server connection failure.

In some implementations, in instances of a server connection failure (e.g., interruption or loss of communication connection between server(s) and one or more sensors and/or monitoring hubs), the one or more sensors 102 and/or the monitoring hubs 100A, 100B can store the unprocessed and/or processed physiological data, such as in a buffer, for later transfer to server(s) 106 when connection is improved or reestablished.

In some implementations, in instances of server connection failure, the server(s) 106 can monitor a subject's physiological condition from stored data, such as data maintained in a buffer. Stored data can include data points received from a physiological sensor and/or monitoring hub such as described herein. For example, previously received sensor data may be maintained such as in the buffer. The data points may optionally include data points corresponding to extrapolated and/or interpolated data points, which may correspond to a time when the server(s) 106 are not receiving physiological data from sensors and/or monitoring hubs. Extrapolated and/or interpolated data points may be generated by the controller and may be generated during and/or after server connection failure. Extrapolated and/or interpolated data points may not have been received from a sensor or monitoring hub. Extrapolated/interpolated data points may undergo the same post-processing as actual data points received from a sensor and/or monitoring hub.

In some implementations, the server(s) 106 device can backfill some or all of the data points at periods of time corresponding to when the server(s) are not receiving data after such periods have already ended. For example, the server(s) can adjust, modify, edit, delete, and/or add data at the periods of time corresponding to server connection failure after connection has improved or been reestablished.

In aspects where data (e.g., physiological parameters) is displayed to a user, the data may be displayed uniformly such that a user may not be able to distinguish between data that was received from sensors and data that was extrapolated and/or interpolated.

Maintaining the buffer can include not deleting, erasing, or discarding one or all of the data in the buffer. Maintaining the buffer can include preserving a state of the buffer or a state of the data in the buffer. Maintaining the buffer can include continuing to perform post-processing, algorithms, or operations to data in the buffer. Maintaining the buffer can include making no changes to data in the buffer and/or processes, operations, or algorithms performed on data in the buffer. The server(s) 106 can maintain the buffer even though no new data values are received. Advantageously, the buffers can help server(s) 106 to preserve physiologically accurate measurements even when physiological data is not being received from one or more physiological sensors and/or monitoring hubs. In some implementations, the server(s) 106 may determine that a physiological alarm condition is satisfies, such as based on physiological data stored in the buffer (e.g., extrapolated and/or interpolated data). Upon reconnecting with the one or more sensors 102 and/or the monitoring hubs 100A, 100B (or upon connection strength increasing), the server(s) 106 may send an alarm signal to the one or more sensors 102 and/or monitoring hubs 100A, 100B to output an alarm, such as described herein.

In some implementations, the server(s) 106 can generate user interface data corresponding to physiological data for rendering user interfaces comprising indicia of the physiological data. Advantageously, the server(s) 106 may act as a central processing computing system that processes data from distributed computing devices (e.g., monitoring hubs 110, sensors 102, user device 107, etc.) which may reduce local processing requirements on the distributed computing devices. In some implementations, the server(s) 106 may store data received from monitoring hub 110A and/or 110B, such as physiological data and/or wireless configuration data. Advantageously, the server(s) 106 may act as a central storage (e.g., database) such that distributed computing devices (e.g., monitoring hubs 110, sensors 102, user device 107, etc.) can access the same data stored at the server(s) 106 which may reduce local storage requirements at the distributed computing devices.

The user device 107 may communicate with the server(s) 106 via the network 104. The user device 107 can communicate with the server(s) 106 via one or more wireless communication protocols such as Wi-Fi. The user device 107 may communicate data to the server(s) 106 including physiological data and/or communication data. The server(s) 106 can communicate data to the user device 107 such as physiological data and/or communication data. The user device 107 may be a phone, laptop, PC, wearable device, smartwatch, tablet, or the like. The server(s) 106 can communicate data to the user device 107 upon request, such as in response to a request from the user device 107. The server(s) 106 can communicate data to the user device 107 periodically. The server(s) 106 can communicate data to the user device 107 in response to one or more conditions. For example, the server(s) 106 may communicate data to the user device 107 in response to a patient being discharged from a healthcare facility. Such data can include historical physiological data previously collected from sensors 102 and communicated to the server(s) 106 from monitoring hub 100A and/or 100B. Advantageously, the user may have access to physiological data collected while the patient was in the healthcare facility which may facilitate continuing to care for the patient when the patient goes home. The user device 107 may be associated with a patient. For example, the user device 107 may belong to the patient or to a person associated with the patient. The server(s) 106 may verify the user device 107 is associated with the patient before communicating patient-specific data to the user device 107.

FIG. 1B is a schematic block diagram illustrating an example implementation of the physiological monitoring system (PMS) 150. The example implementation shown in FIG. 1B may result from a request to transfer physiological monitoring from monitoring hub 100A to monitoring hub 100B. Transferring physiological monitoring from one monitoring hub to another monitoring hub is described in greater detail in U.S. patent application Ser. No. 18/584,550, filed on Feb. 22, 2024, entitled “PHYSIOLOGICAL MONITORING DEVICES, SYSTEMS, AND METHODS,” the entire contents of which are hereby incorporated herein by reference. For example, as shown in the example implementation of FIG. 1A, the sensor(s) 102 may be in communication with the monitoring hub 100A and may not be in communication with the monitoring hub 100B. The PMS 150 can transfer physiological monitoring (e.g., in response to a user request) from the monitoring hub 100A to the monitoring hub 100B. As shown in FIG. 1B, after the PMS 150 has transferred the physiological monitoring from monitoring hub 100A to monitoring hub 100B, the sensor(s) 102 may be in communication with the monitoring hub 100B and may not be in communication with the monitoring hub 100A. The monitoring hub 100B can receive and/or display physiological data received from the sensor(s) 102 via the wireless communication connection (e.g., Bluetooth and/or other wireless communication protocol) established with the sensor(s) 102 as a result of the transfer.

The monitoring hubs 100A, 100B can receive a user input 108. The user input 108 can include identification data and/or a transfer request. The user input 108 can be a manual user input such as via a display of the monitoring hubs 100A, 100B or via one or more buttons of the monitoring hubs 100A, 100B. For example, a user may press a button of the monitoring hubs 100A, 100B to request a transfer. The user input 108 can include an electronic input such as an electronic signal generated in response to a wireless communication protocol. For example, a user may bring a communication device (e.g., user ID badge) in proximity to the monitoring hubs 100A, 100B to generate an electronic signal (e.g., via NFC and/or RFID) at the monitoring hubs 100A, 100B.

In some implementations, the monitoring hub 100A can optionally communicate with the monitoring hub 100B. In some implementations the monitoring hub 100A may communicate with the monitoring hub 100B via any variety of communication protocols such as Wi-Fi (e.g., 2.4 GHz channel, 5 GHz channel), Bluetooth® (e.g., Bluetooth Low Energy 5.0/Mesh), ZigBee®, Z-wave®, cellular telephony, 1G, 2G, 3G, 4G, 5G, infrared, near-field communication (NFC), radio frequency identification (RFID), satellite transmission, inductive coupling, capacitive coupling, proprietary protocols, any combination of the foregoing, and/or any other suitable wireless connection. The monitoring hub 100A may communicate data to the monitoring hub 100B and/or receive data from the monitoring hub 100B including communication data (e.g., device addresses of the sensors 102), physiological data, identification data (e.g., user ID), transfer requests, request approval status, or the like. In some implementations, the monitoring hub 100A may communicate with the monitoring hub 100B only while the PMS 150 is transferring the physiological monitoring from the monitoring hub 100A to the monitoring hub 100B. For example, in some implementations, the monitoring hub 100A may only communicate with the monitoring hub 100B until the transfer is complete, the monitoring hub 100B has established communication with the sensor(s) 102, or the like. In some implementations, the monitoring hub 100A may communicate with the monitoring hub 100B to facilitate the transfer (e.g., may transmit communication data to facilitate establishing communication between the monitoring hub 100B and the sensor(s) 102. In some implementations, the monitoring hub 100A may not communicate with the monitoring hub 100B. In some implementations, the monitoring hub 100B may receive data from the server(s) 106, such as wireless configuration data and/or physiological data which may facilitate transferring physiological monitoring to the monitoring hub 100B.

As shown in FIGS. 1A-1B, the server(s) 106 can receive audio data from monitoring hub 100A and/or monitoring hub 100B such as via one or more wireless communication protocols (e.g., Wi-Fi) over network 104. For example, monitoring hub 100A and/or 100B may detect audio with one or more microphones and may communicate the detected audio to the server(s) 106. In some implementations, monitoring hub 100A and/or 100B may detect (e.g., continuously) ambient noise and may communicate the ambient noise to the server(s) 106. In some implementations, the monitoring hub 100A and/or 100B may communicate processed and/or unprocessed audio data to the server(s) 106, such as raw acoustic signals detected with a microphone and/or representations of acoustic signals such as decibel levels detected. Advantageously, in some implementations, the monitoring hub 100A and/or 100B may only communicate decibel levels of detected audio (e.g., without communicating underlying audio signals) to the server(s) which may allow the server(s) to access information relating to general noise level in an environment while preventing the server(s) from accessing sensitive information, such as audio data of a conversation.

The monitoring hubs 100A and 100B may be at different locations from each other and may change locations as they travel about an environment, such as a healthcare facility. As the monitoring hubs 100A and 100B travel throughout an environment they can detect and communicate (e.g., continuously) audio data to the server(s) 106 that they detect with microphone(s). The server(s) 106 can access audio data detected by and/or communicated from monitoring hub 100A and/or 100B. The server(s) 106 can process the audio data to determine an audio map of an environment. An audio map can indicate noises and/or noise levels associated with locations of an environment, such as particular rooms, hallways, floors, etc. of a building. For example, an audio map can indicate that a particular location, such as room, has an average decibel level. As another example, an audio map can indicate that a particular location generally has particular sounds such as alarms, people speaking, equipment operating, etc. An audio map may represent historical audio-spatial data, current audio-spatial data, and/or predictive future audio-spatial data. The server(s) 106 can determine a location for a patient based on an audio map generated using audio data from the monitoring hub 100A and/or 100B as well as patient characteristics such as health condition, age, diagnosis, scheduled procedure, medication regimen, or the like. For example, the server(s) 106 can determine that a patient having a certain health condition should be in a location with a low noise level (e.g., below a certain decibel threshold) to improve their health and the server(s) 106 can determine a location that has historical met, is currently meeting, or is predicted to meet that noise level criteria. In some implementations, the server(s) 106 can determine locations for patients based on decibel level and/or noise types. For example, the server(s) 106 can determine that a patient should avoid being in certain locations having frequent alarms or frequent human conversations because these types of noise may be particularly disturbing to a patient (although perhaps below a certain decibel threshold), whereas a constant low-frequency noise from a machine that is operating may not disturb a patient (although perhaps above a decibel threshold).

FIG. 1C illustrates an example implementation of a physiological monitoring system (PMS) including a monitoring hub 110A, a monitoring hub 110B, and one or more sensors 122. The PMS, or portions thereof, shown and discussed in FIG. 1C can include similar structural and/or operational features as the PMS 150, or portions thereof, shown and/or discussed in FIGS. 1A-1B. For example, the monitoring hub 110A can include similar structural and/or operational features as the monitoring hub 100A discussed in FIGS. 1A-1B. In some examples, the monitoring hub 110B can include similar structural and/or operational features as the monitoring hub 100B discussed in FIGS. 1A-1B. In some examples, the one or more sensors 122 can include similar structural and/or operational features as the sensor(s) 102 discussed in FIGS. 1A-1B.

As shown in this example implementation, the monitoring hub 110A is monitoring the physiological data of the subject 111 and the monitoring hub 110B is not monitoring the physiological data of the subject 111.

The one or more sensors 122 can be configured to obtain physiological data of the subject 111. The monitoring hub 110A is in electrical communication with the one or more sensors 122. In some implementations, the electrical communication between the monitoring hub 110A and the one or more sensors 122 may include a wireless communication protocol such as Bluetooth. The monitoring hub 110A receives physiological data from the one or more sensors 122 (e.g., in real-time as the data is generated by the one or more sensors 122). The monitoring hub 110A displays indicia of the physiological data and/or information relating thereto on a display of the monitoring hub 110A.

The monitoring hub 110B is not in electrical communication with the sensor 122. The monitoring hub 110B does not receive or display physiological data from the sensor 122. A user, such as a healthcare provider (e.g., doctor, nurse, etc.) may desire to transfer physiological monitoring from the monitoring hub 110A to the monitoring hub 110B. As described in greater detail in U.S. patent application Ser. No. 18/584,550, the user can transfer physiological monitoring from the monitoring hub 110A to the monitoring hub 110B such that the monitoring hub 110B would receive and display physiological data from the one or more sensors 122 and monitoring hub 110A would discontinue to receive and/or display physiological data from the one or more sensors 122. Subsequent to transferring physiological monitoring, the monitoring hub 110B could display indicia of physiological data previously displayed on the monitoring hub 110A. The monitoring hub 110B could display a user interface similar or identical, in whole or in part, to a user interface previously displayed by the monitoring hub 110A.

The one or more sensors 122 can include any number and/or type of sensors. The one or more sensors 122 can include an auricular device 123, such as an earbud, earpiece, or the like. The one or more sensors 122 can include an ECG device 113 which may include and/or be coupled to one or more ECG electrodes 112. The one or more sensors 122 can include a temperature sensor 114. The one or more sensors 122 can include a motion sensor 115 which can include one or more of a position sensor, motion sensor, gyroscope, accelerometers, or the like. The one or more sensors 122 can include an acoustic sensor 116. The one or more sensors 122 can include a wearable device 118, such as a smart device, which can comprise a watch. The wearable device 118 can include one or more sensors such as a pulse oximeter (including an optical emitter and optical detector), a temperature sensor, and/or one or more electrodes configured for measuring electrical signals for electrocardiogram some implementations, the wearable device 118 may not include a pulse oximeter and/or may not measure blood oxygen saturation. The one or more sensors 122 can include a pulse oximeter such as optical sensor 140 which can comprise a finger sensor. The one or more sensors 122 can include a blood pressure monitor 121. The one or more sensors 122 can include a wearable hub 130. The wearable hub 130 may be coupled to one or more of the sensors shown and/or described herein. The wearable hub 130 may be in communication with one or more of the sensors shown and/or described herein. The wearable hub 130 may receive data from one or more of the sensors shown and/or described herein. In some implementations, the wearable hub 130 may be in communication with monitoring hub 110A and/or monitoring hub 110B. In some implementations, wearable hub 130 may collect sensor data from one or more sensors and communicate the sensor data to monitoring hub 110A and/or monitoring hub 110B. In some implementations, one or more of the sensors shown and/or described herein, may communicate directly with the monitoring hub 110A and/or monitoring hub 110B. For example, the wearable device 118, auricular device 123, optical sensor 140, ECG device 113, acoustic sensor 116, temperature sensor 114, motion sensor 115, blood pressure monitor 121 may communicate directly with the monitoring hub 110A and/or monitoring hub 110B, such as via wireless communication. In some implementations, the one or more sensors can include one or more of an infusion pump, a brain monitoring device, a depth of conscience device, or a pacemaker. In some implementations, the one or more sensors 122 can include a capnography device (e.g., associated with a respiratory device coupled to the subject's 111 mouth) configured to measure concentration of carbon dioxide in air that is exhaled from the subject 111. In some implementations, the one or more sensors 122 can include a transducer coupled to an arterial line configured to measure hemodynamic parameters such as mean arterial pressure, cardiac output, stroke volume, heart rate, systemic vascular resistance, stroke volume variation, pulse pressure variation, oxygen delivery, oxygen consumption, and/or heart rate variation.

The one or more sensors 122 can include an EEG sensor 117 which may attach to the subject's 111 forehead, and which may be responsive to electrical signals originating from the subject's 111 brain. The EEG sensor 117 can monitor brain function, including measuring depth-of-consciousness (e.g., under anesthesia). The one or more sensors 122 can include an oximetry sensor 109. The oximetry sensor 109 may perform region-specific oximetry. The oximetry sensor 109 can be attached to the subject's 111 forehead.

One or more of the sensors 122, such as the EEG sensor 117 and/or oximetry sensor 109 can connect to a portable monitoring hub 119. The portable monitoring hub 119 may also be referred to as a transport device. The portable monitoring hub 119 may receive physiological data originating from the one or more sensors 122 via a wired connection. The portable monitoring hub 119 may comprise one or more ports 124 which may be Mach ports (e.g., Mach 9). A Mach port may be a unidirectional channel that may have multiple send endpoints and a single receive endpoint. A Mach port may be a kernel-provided inter-process communication (IPC) mechanism. The portable monitoring hub 119 can receive physiological data via the one or more ports. In some implementations, the portable monitoring hub 119 can receive, via the ports, one or more of capnography data, depth-of-consciousness data, sedation data, oximetry data such as regional oximetry data, or hemodynamic data. In some implementations, the portable monitoring hub 119 may receive physiological data originating from the one or more sensors 122 via a wireless communication protocol. The portable monitoring hub 119 can comprise one or more hardware processors configured to execute program instructions such as to process physiological data originating from the one or more sensors 122. The portable monitoring hub 119 may comprise a display 120 which can display indicia of physiological data including processed physiological parameters, trend lines, graphs, icons, alarms, notifications, or the like. The portable monitoring hub 119 may couple to a bed 125 such as to a rail 126 of the bed 125, a frame of the bed 125, or the like. The portable monitoring hub 119 can include a battery. The portable monitoring hub 119 can include a port for receiving power from an external source, such as AC power.

The portable monitoring hub 119 can communicate data, such as physiological data, to the monitoring hub 110A and/or 110B. The portable monitoring hub 119 can communicate data to the monitoring hub 110 via a wireless communication protocol such as Bluetooth or Wi-Fi. The portable monitoring hub 119 can communicate with the monitoring hub 110 via Wi-Fi directly (e.g., without the use of intermediary routers). Communicating via Wi-Fi may allow for transmission of data at a higher frequency rate than Bluetooth. In some implementations, the portable monitoring hub 119 may establish communication with monitoring hub 110 based on proximity to the monitoring hub 110. For example, the portable monitoring hub 119 may establish wireless communication with monitoring hub 110 when a distance between portable monitoring hub 119 and the monitoring hub 110 is less than a threshold. The monitoring hub 110 can display indicia of physiological data received from the portable monitoring hub 119. The portable monitoring hub 119 can communicate data, such as physiological data, to a remote server via wireless communication protocol such as Wi-Fi. In some implementations, the portable monitoring hub 119 may establish communication with a remote server based on proximity to the monitoring hub 110. For example, the portable monitoring hub 119 may establish wireless communication with a remote server when a distance between the portable monitoring hub 119 and a monitoring hub 110 is greater than a threshold and/or when the portable monitoring hub 119 has diminished or no communication with the monitoring hub 110. The portable monitoring hub 119 can receive data, such as physiological data, from a remote server via wireless communication protocol such as Wi-Fi.

The portable monitoring hub 119 may determine distance to a monitoring hub 110 and/or quality of a wireless communication connection with a monitoring hub 110 based on a signal strength between the portable monitoring hub 119 and the monitoring hub 110. The portable monitoring hub 119 can display indicia of physiological data based on distance to a monitoring hub 110 and/or quality of a wireless communication connection with a monitoring hub 110 (e.g., based on a signal strength). For example, the portable monitoring hub 119 may display indicia of physiological data only not in proximity to and/or communicating with a monitoring hub 110.

The monitoring hub 110A is shown as being in a fixed location (e.g., mounted to the wall). The monitoring hub 110A may be removably mounted to the wall. The monitoring hub 110B is shown as resting on a surface adjacent to the subject 111 and may be portable or mobile (e.g., not fixed to a particular location). The monitoring hub 110B is housed within a holder 132. The holder 132 is configured to support the monitoring hub in an upright position. In some implementations, the monitoring hub 110B may be fixed or removably fixed to a particular location. For example, the monitoring hub 110B may couple to a structure, such as a bed, by the holder 132. In some implementations, the monitoring hub 110A may be portable or mobile. In some implementations, the monitoring hub 110A may be housed within a holder similar or identical to the holder 132.

FIG. 1D is a schematic block diagram illustrating an implementation of an example physiological monitoring system (PMS) 160. The PMS 160 may include similar features as other PMS discussed herein such as PMS 150 shown and/or discussed with respect to FIGS. 1A-1B. The operations, processes, functionality of the PMS 160 may be performed by one or more computing devices (e.g., hardware processor(s) of computing devices) such as any of the computing devices discussed herein, such as monitoring hub(s), sensor(s), and/or server(s), for example.

The PMS 160 can receive one or more inputs. The PMS 160 can receive a transfer request 162. The PMS 160 can receive the transfer request 162 via one or more monitoring hubs as described in greater detail, such as in U.S. patent application Ser. No. 18/584,550, for example. The PMS 160 can receive identification data 164. The PMS can receive the identification data 164 via one or more monitoring hubs as described in U.S. patent application Ser. No. 18/584,550, for example.

The PMS 160 can receive and/or access physiological data 166. The PMS 160 can receive the physiological data 166 from one or more physiological sensors. In some implementations, a monitoring hub of the PMS 160 can receive the physiological data 166 in real-time and/or directly from the one or more sensors. In some implementations, a server of the PMS 160 can receive the physiological data 166 in real-time and/or directly from the one or more sensors. In some implementations, a monitoring hub of the PMS 160 can transmit the physiological data 166 to a server of the PMS 160 after having received it from the sensors. The server can determine one or more alarm conditions based on the physiological data 166. The server can generate one or more alarms based on the physiological data 166 and the one or more alarm conditions. The server may send alarm signals indicative of an alarm to the one or more sensors and/or the monitoring hub for output of the alarm, such as described herein.

The PMS 160 can receive and/or access communication data 168. Communication data can include data to facilitate establishing communication between computing devices. Communication data can include device addresses of computing devices such as device addresses of monitoring hubs and/or physiological sensors, for example. The PMS 160 can access communication data 168 of a computing device from the computing device. The PMS can transmit communication data 168 between computing devices of the PMS 160 such as between monitoring hubs, sensors, and/or servers.

The PMS 160 can process the one or more inputs (e.g., inputs 162, 164, 166, 168) to generate an output. The PMS can process the inputs as described in greater detail in U.S. patent application Ser. No. 18/584,550, for example. The PMS 160 can output a transfer physiological monitoring operation 169 in response to receiving and/or processing one or more inputs. The transfer physiological monitoring operation 169 can include transferring physiological monitoring from one monitoring hub of the PMS 160 to another monitoring hub of the PMS 160. Transferring physiological monitoring can include transferring output of an alarm between computing devices of the PMS 160, such as between monitoring hubs and/or sensors. The transfer physiological monitoring operation 169 may be described in greater detail in U.S. patent application Ser. No. 18/584,550, for example.

### Example Implementations

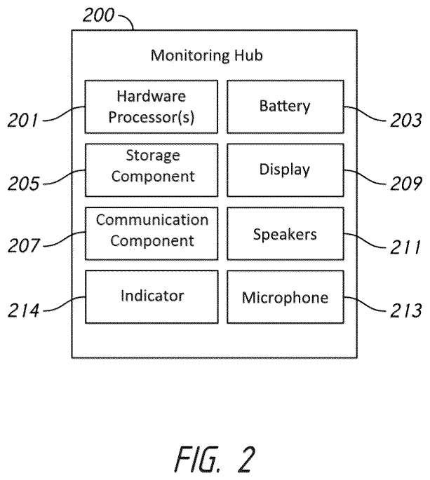

FIG. 2 is a block diagram illustrating an example implementation of a monitoring hub 200. The monitoring hub 200 can include similar structural and/or operational features as any of the other example monitoring hubs shown and/or discussed herein such as monitoring hubs 100A, 100B discussed in FIG. 1A.

As shown, the monitoring hub 200 can include a hardware processor 201, a storage component 205, a communication component 207, and a battery 203. The hardware processor 201 can be configured, among other things, to process data, execute program instructions to perform one or more functions, and/or control the operation of the monitoring hub 200. For example, the hardware processor 201 can process physiological data obtained from physiological sensors and can execute instructions to perform functions related to storing and/or transmitting such physiological data. In some examples, the hardware processor 201 can process data relating to transfer requests, identification data, and/or transfer approval status. In some examples, the hardware processor 201 can process alarm configuration data and can execute instructions to perform functions related to generating and outputting alarms. In some implementations, the hardware processor 201 can determine the quality of a communication connection with a remote server. For example, the hardware processor 201 can determine that communication connection with a remote server has been interrupted or lost, or has been improved or reestablished.

The storage component 205 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. The storage component 205 can store data including processed and/or unprocessed physiological data originating from physiological sensors. The storage component 205 can store data including alarm configuration data, such as alarm configuration data originating from a remote server. The storage component 205 can store data including communication data such as link keys and/or device addresses associated with sensors and/or monitoring hubs. The storage component 205 can store persistent data and/or non-persistent data. Persistent data may be data that is preserved (e.g., not deleted from storage) when the computing system is powered down and/or when an application is terminated. Persistent data may be stored for longer than 30 seconds, longer than 60 seconds, longer than 5 minutes, longer than 10 minutes, longer than 30 minutes, longer than 1 hour, longer than 6 hours, longer than 12 hours, longer than 24 hours, or the like. Non-persistent data may be data that is deleted or otherwise lost when the computing system is powered down and/or when an application is terminated. Non-persistent data may be stored for less than 24 hours, less than 12 hours, less than 6 hours, less than 1 hour, less than 30 minutes, less than 10 minutes, less than 5 minutes, less than 60 seconds, less than 30 seconds, less than 20 seconds, less than 10 seconds, less than 5 seconds, less than 1 seconds, or the like. Non-persistent data may be stored for a shorter period of time than persistent data. In some implementations, persistent data may be stored in non-volatile memory. In some implementations, non-persistent data may be stored in volatile memory such as RAM. The storage component 205 can store data in a buffer. A buffer may store data for a period of time before deleting the data. The period of time can be fixed. The buffer may automatically delete data stored therein upon expiration of a period of time. The period of time may be between about 0.01 seconds and 0.15 seconds, between 0.1 seconds and 1.5 seconds, between 1 second and 5 seconds, between 1 second and 10 seconds, between 10 seconds and 60 seconds, between 30 seconds and 60 seconds, between 1 minute and 3 minutes, between 1 minute and 5 minutes, between 1 minute and 10 minutes, between 5 minutes and 30 minutes, between 20 minutes and 60 minutes, or greater than 60 minutes.

The communication component 207, which may also be referred to as a communication system, can facilitate communication (via wired and/or wireless connection) between the monitoring hub 200 (and/or components thereof) and separate computing devices, such as separate monitoring hubs, monitoring devices, sensors, systems, servers, or the like. For example, the communication component 207 can be configured to allow the monitoring hub 200 to wirelessly communicate with other devices, systems, using any combination of a variety of communication protocols and/or over one or more networks. The communication component 207 can be configured to implement any combination of a variety of wireless communication protocols, such as Wi-Fi (e.g., 802.11x), Bluetooth®, ZigBee®, Z-wave®, cellular telephony, 1G, 2G, 3G, 4G, 5G, infrared, near-field communications (NFC), radio frequency identification (RFID), satellite transmission, inductive coupling, capacitive coupling, proprietary protocols, combinations of the foregoing, and the like. The communication component 207 can allow data and/or instructions to be transmitted and/or received to and/or from the monitoring hub 200 and separate computing devices. The communication component 207 can be configured to transmit and/or receive (e.g., wirelessly) processed and/or unprocessed physiological data with separate computing devices including physiological sensors, other monitoring hubs, remote servers, or the like. In some implementations, the communication component 207 can be configured to transmit and/or receive alarm configuration data and/or alarm signals (e.g., wirelessly) with separate computing devices including physiological sensors, other monitoring hubs, remote servers, or the like. In some implementations, the communication component 207 can be configured to transmit and/or receive (e.g., wirelessly) communication data (e.g., link keys and/or device addresses associated with monitoring hubs and/or sensors) with separate computing devices including physiological sensors, other monitoring hubs, remote servers, or the like. The communication component 207 can be embodied in one or more components that may be in communication with each other. The communication component 207 can include one or more wireless transceivers, one or more antennas, one or more radios, and/or a near field communication (NFC) component such as a transponder. The communication component 207 can wirelessly communicate or connect to one or more remote computing devices over a network such as by implementing one or more wireless communication protocols.

The monitoring hub 200 can include a battery 203. The battery 203 can provide power for hardware components of the monitoring hub 200 described herein. The battery 203 can be, for example, a lithium battery. Additionally or alternatively, the monitoring hub 200 can be configured to obtain power from a power source that is external to the monitoring hub 200. For example, the monitoring hub 200 can include or can be configured to connect to a cable which can itself connect to an external power source to provide power to the monitoring hub 200.

The monitoring hub 200 can include a display 209. The display 209 can include an LED screen, an LCD screen, an OLED screen, a QLED screen, a plasma display screen, a quantum dot display screen, or the like. The display 209 may be responsive to touch. For example, the display screen may comprise a touchscreen such as a resistive touchscreen, a capacitive touchscreen, an infrared touchscreen, a surface acoustic wave touchscreen, or the like. The display 209 can receive user input. The display 209 can render one or more user interfaces. The display 209 can display indicia of physiological data. The display 209 can display indicia of an alarm.

The monitoring hub 200 can include one or more speakers 211. The speakers 211 can emit an audio signal. The speakers 211 can emit an alarm. The speakers 211 can emit a voice audio signal. The speakers 211 can include a plurality of speakers positioned apart from one another at various positions on the monitoring hub 200. The speakers 211 can emit stereophonic audio. The speakers 211 can emit audio using one or more audio channels. The speakers 211 can emit audio in a plurality of directions. The speakers 211 can emit monaural audio. The speakers 211 can emit audio originating during a voice call and/or video call.

The monitoring hub 200 can include one or more microphones 213. The microphone 213 can detect audio signals and generate signals responsive to the detected audio signals. The microphone 213 can detect ambient noise. The microphone 213 can detect a noise level in an environment surrounding the monitoring hub 200. The microphone 213 can detect a noise level in an environment adjacent to and/or encompassing a subject. The monitoring hub 200 can adjust one or more operations based on at least an ambient noise level detected by the microphone 213. The monitoring hub 200 can perform one or more operations to increase a patient's comfort based on at least an ambient noise level detected by the microphone 213. The microphone 213 can detect the voice a person speaking. The microphone 213 can detect a person's voice during a voice call and/or during a video call. In some implementations, the monitoring hub 200 can include two microphones 213. One of the microphones can detect ambient noise and another of the microphones 213 can detect a person's voice. The hardware processor 201 can perform noise cancelling based on audio detected by the microphones 213. For example, the hardware processors 201 may cancel (e.g., suppress, subtract, reduce, etc.) ambient noise detected with one microphone from audio (e.g., a person's voice) detected with another microphone, which may greatly improve the audio quality of the person's voice such as during audio calls with the monitoring hub 200. In some implementations, the monitoring hub 200 can communicate audio detected with the microphone(s) 213 to a remote computing device via communication component 207. For example, the monitoring hub 200 (with communication component 207) can communicate audio detected with the microphone 213 to a remote computing device (e.g., another monitoring hub, phone, laptop, etc.) during an audio call. As another example, the monitoring hub 200 can communicate audio detected with the microphone 213 to a remote server which may determine an audio map of an environment from the audio communicated from the monitoring hub 200 and/or from other monitoring hubs. In some implementations, the microphone 213 may continuously monitor an ambient noise and may communicate the ambient noise to a remote server.

The monitoring hub 200 can implement a voice call. The monitoring hub 200 can connect to one or more cellular devices. The monitoring hub 200 can implement a voice call using cellular telephony such as via the communication component 207. The monitoring hub 200 can implement a video call. A patient being monitored by the monitoring hub 200 can talk to another person in a remote location, such as a caregiver, via the monitoring hub 200 which can implement one or more wireless communication protocols, such as mobile telephony, to connect to one or more remote computing devices over a network, and which can detect the patient's voice using the microphone 213.

The monitoring hub 200 can include one or more indicators 214. The indicator 214 can include a visual indicator. The indicator 214 can include an LED indicator, comprising one or more LEDs. The indicator 214 can emit one or more visual signals. The visual signals may correspond to a physiological status of a patient being monitored by the monitoring hub 200. The indicator 214 can emit visual signals including a plurality of colors. The indicator 214 can emit visual signals according to a color-code scheme wherein various colors may correspond to various physiological statuses of a patient being monitored by the monitoring hub 200.

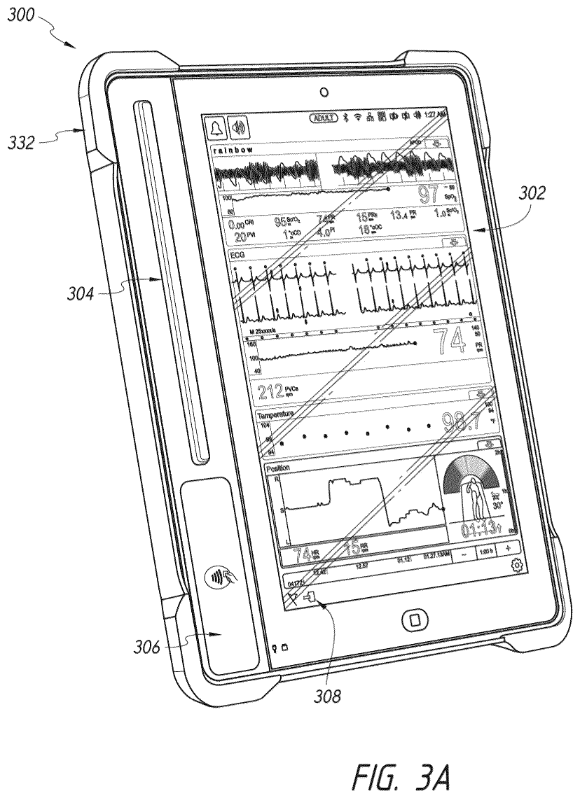

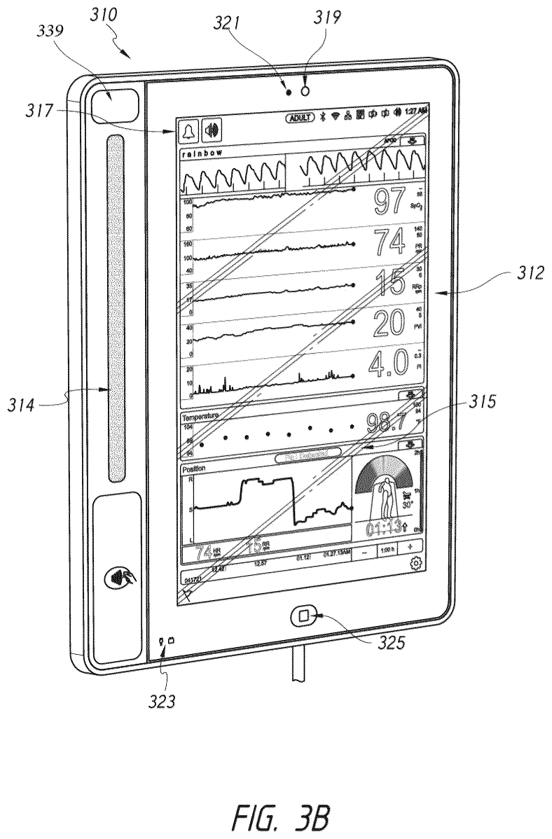

FIG. 3A is a perspective view of an example monitoring hub 300. Monitoring hub 300 can include similar structural and/or operational features as any of the other example monitoring hubs shown and/or described herein. In this example implementation, the monitoring hub 300 is secured within a holder 332. The monitoring hub 300 may be removably secured within the holder 332, for example, via a friction fit. In this example implementation, the monitoring hub 300 can include a display 302. The display 302 can include an LED display. The display 302 can include a touchscreen configured to receive user input in response to touching the display 302. The display 302 can display indicia of physiological data, including physiological trends, graphs, graphics, charts, parameters, values, percentages, animations, visualizations, alarms, alerts, notifications, and the like. The display 302 can display indicia of physiological data from a plurality of sensors including different types of sensors that measure different types of physiological data. The monitoring hub can generate (and/or receive) user interface data for rendering the display indicia based on at least physiological data originating from one or more sensors or other devices.

The monitoring hub 300 can include a status indicator 304, such as described in greater detail in U.S. patent application Ser. No. 18/584,550. The status indicator 304 can include one or more LEDs. The status indicator 304 can indicate a status of a subject being monitored by the monitoring hub 300. For example, the status indicator 304 may illuminate in response to a certain change in the physiological data of the subject, such as physiological parameter(s) of the subject, satisfying an alarm condition (e.g., exceeding a threshold). The status indicator 304 may illuminate different colors during different operations or modes. The status indicator 304 may illuminate (e.g., red) during an alarm mode. The status indicator 304 may not illuminate when the monitoring hub 300 is not in alarm mode. In some implementations, the monitoring hub 300 can assign one or more LEDs of the status indicator 304 to each of different physiological parameters displayed and/or measured by the monitoring hub 300. For example, each physiological parameter capable to being displayed and/or measured by the monitoring hub 300 may be associated with a group of LEDs, such as distinct groups of LEDs. When the monitoring hub 300 generates or receives an alarm (such as an alarm signal from a remote server) associated with a certain physiological parameter, the monitoring hub 300 may cause the group of LEDs associated with the physiological parameter to illuminate. LEDs not associated with the physiological parameter may not illuminate.

The monitoring hub 300 can include a communication interface 306. The communication interface 306 can include electronics configured to execute a wireless communication protocol. The communication interface 306 can include an NFC and/or RFID transponder or reader. The communication interface 306 can include a bar code reader or scanner. The communication interface 306 can include a QR code reader or scanner. The communication interface 306 can include a fingerprint scanner. The communication interface 306 can include a camera configured to capture images of a user's face for facial recognition. The communication interface 306 can use magnetic field induction to communicate with a separate device. The communication interface 306 can communicate with a user device such as a user ID badge, a user phone, a user mobile device, a user smartwatch, or the like, to identify and/or verify a user such as by receiving a unique user identification from the user device.

The monitoring hub 300 can include a transfer request button 308. The transfer request button 308 can include a capacitive sensor configured to generate one or more signals in response to a user's touch. The transfer request button 308 can include one or more physical or mechanical actuators configured to generate one or more signals in response a physical actuation of the button 308, such as depression of the button 308. A user may press the transfer request button 308 to initiate transfer of physiological monitoring to the monitoring hub 300 or from the monitoring hub 300. In some implementations, the transfer request button 308 can be incorporated into the display 302 (e.g., as part of a touchscreen display).

FIG. 3B is a perspective view of an example monitoring hub 310. Monitoring hub 310 can include similar structural and/or operational features as any of the other example monitoring hubs shown and/or described herein. Monitoring hub 310 can include a light sensor 319, a microphone 321, one or more indicators 323, a display 312, and/or a button 325. The light sensor 319 can be configured to detect an ambient light. The monitoring hub 310 may change a display brightness of the display 312 based on the ambient light detected by the light sensor 319. In some implementations, the light sensor 319 can comprise a camera configured to capture images. The microphone 321 can be configured to detect sound. In some implementations, the monitoring hub 310 may receive user input such as a transfer request as a voice command via a microphone 321. In some implementations, the monitoring hub 310 may implement voice recognition on sounds detected by the microphone 321. The indicator(s) 323 may include one or more LEDs. The indicator(s) 323 may indicate a status and/or operational state of the monitoring hub 310 such as power level, state of wireless connection, or the like. A user may operate the button 325 to control operation of the monitoring hub 310.

In this example implementation, the monitoring hub 310 is in alarm mode. During alarm mode, a status indicator 314 may illuminate (e.g., red). Light emitted by the status indicator 314 may be seen from multiple different viewing angles, such as further described in U.S. patent application Ser. No. 18/584,550. During alarm mode, a display 312 of the monitoring hub 310 may display one or more badges, icons, banners, symbols, indicators, or the like to indicate the monitoring hub 310 is in alarm mode. During this example alarm mode, the display 312 can include a “Fall Detected” banner 315. During this example alarm mode, the display 312 illuminates an alarm icon 317.

In some implementations, monitoring hub 310 can include an alarm toggle button 339. A user may press the alarm toggle button 339 to silence an alarm. A user may press the alarm toggle button 339 to change a state of an alarm. The alarm toggle button 339 can comprise a capacitive sensor. The alarm toggle button 339 can comprise one or more mechanical actuators.

### Example Methods

FIG. 4 is a flowchart illustrating an example process 400 of generating an alarm by a physiological sensor and/or monitoring hub, such as when there is connection failure (e.g., interrupted or lost communication connection) with, respectively, a monitoring hub and/or a remote server. In some implementations, the physiological sensor can include a display configured to, among other things, render and cause display of visual indicia indicative of an alarm. In some implementations, the physiological sensor may not include a display. The physiological sensor can be equipped with an audio device and/or status indicator (e.g., LEDs) configured to indicate an alarm, such as be outputting audio and/or emitting light, respectively.

One or more hardware processors can execute process 400, or portions thereof. Process 400, or portions thereof, can be implemented on one or more computing devices described herein, such as a physiological sensor, an origin monitoring hub, a destination monitoring hub, a server, etc. Process 400, or portions thereof, may be executed by one or more hardware processors of a single computing device. Process 400, or portions thereof, may be executed by one or more hardware processors of multiple computing devices such as computing devices that are remote to each other and/or in wireless communication with each other. In some implementations, one or more hardware processors associated with a physiological sensor, such as physiological sensor 102 shown and/or described herein, may execute process 400, or portions thereof. In some implementations, one or more hardware processors associated with a monitoring hub, such as monitoring hub 100A and/or monitoring hub 100B shown and/or described herein, may execute process 400, or portions thereof. In some implementations, one or more hardware processors associated with a server, such as server 106 shown and/or described herein, may execute process 400, or portions thereof. Process 400 is provided as an example and is not intended to be limiting of the present disclosure. In some implementations, one or more hardware processors executing the process 400 may omit portions of the process 400, may add additional operations, and/or may rearrange an order in which the operations of the process 400 are executed.

At block 401, a physiological monitoring device (e.g., sensors 102, monitoring hub 100A, 100B, etc.) can receive sensor data. Sensor data can include any of the sensor data described herein. For example, sensor data can include physiological data, such as physiological data originating from one or more physiological sensors. In some examples, sensor data can include raw (e.g., unprocessed, unfiltered) data. In some examples, sensor data can include processed and/or filtered data, such as physiological parameters. In some examples, sensor data received at block 401 may correspond to data gathered at a time corresponding to when data is not being transmitted to a remote device, such as a remote server (e.g., during server connection failure). In some examples, sensor data received at block 401 may correspond to data gathered at a time corresponding to when data is being transmitted to a remote device, such as a remote server. In some examples, the sensor data may correspond to interpolated and/or extrapolated data.

In some implementations, at block 401, the monitoring device may be in communication with a physiological sensor, such as a wearable sensor (e.g., a wearable device). The monitoring device may be in communication with any available type of physiological sensor, such as any of the physiological sensors described herein. The physiological sensor can include an emitter having one or more LEDs. The LEDs may be configured to emit light at one or more wavelengths at a measurement site of a subject (e.g., a wearer, a patient, etc.). The physiological sensor can include a detector configured to detect one or more wavelengths of the emitted light. The detector may be configured to generate a physiological signal associated with the light after attenuation by tissue at the measurement site of the subject. The physiological signal may be indicative of a physiological parameter. In some implementations, at block 401, the physiological sensor may be embodied by the monitoring device. In some implementations, at block 401, the physiological sensor may be remote from the monitoring device.

At block 403, the monitoring device can establish communication connection (e.g., wirelessly) with a remote device, such as a remote server. The monitoring device may be in communication with a communication component, such as described herein. The communication component can be configured to permit wireless communication between the monitoring device and the remote device. For example, the communication component can be configured to implement any combination of a variety of available wireless communication protocols, such as Wi-Fi, Bluetooth®, ZigBee®, Z-wave®, cellular telephony, 1G, 2G, 3G, 4G, 5G, infrared, near-field communications (NFC), radio frequency identification (RFID), satellite transmission, inductive coupling, capacitive coupling, proprietary protocols, combinations of the foregoing, and the like. The communication component can be configured to transmit and receive (e.g., wirelessly) processed and/or unprocessed physiological data with the remote device. In some implementations, at block 403, the communication component can be configured to transmit and/or receive alarm configuration data and/or alarm signals (e.g., wirelessly) with the remote device. The communication component may be embodied by the monitoring device. In some implementations, at block 403, the communication component may be remote from the monitoring device.

At block 405, the monitoring device can determine whether communication connection with the remote device is interrupted or lost. In some implementations, at block 504, the monitoring device may determine a quality of the wireless connection, such as based on a signal strength of the wireless connection. For example, the monitoring device may be configured to measure a received signal strength indicator (RSSI) indicative of the power level (typically measured, for example, in dBm) of a wireless signal received by the monitoring device from the remote device. An RSSI farther away from 0 can be indicative of a weak signal, whereas an RSSI closer to or at 0 can indicate a strong signal. The monitoring device may sample (e.g., continuously) the signal's power as it's being received and calculate the RSSI. The monitoring device may compare the RSSI to a signal strength threshold, and determine based on the comparison whether connection with the remote device is interrupted or lost. For example, the monitoring device may determine that the communication connection with the remote device is interrupted or lost based on a determination that the RSSI is less than the signal threshold. In some implementations, at block 405, the monitoring device can be configured to determine other signal strength metrics, including but not limited to, reference signal received power (RSRP), reference signal received quality (RSRQ), and the like.

In some implementations, at block 405, the monitoring device may determine a signal to noise ratio (SNR) of a wireless signal received by the monitoring device from the remote device. A high SNR may be indicative of an uninterrupted communication connection between the monitoring device and the remote device, whereas a low SNR may be indicative of an interrupted or lost communication connection between the monitoring device and the remote device. The monitoring device may be configured to compare the strength of the received signal with noise levels in the frequency band being used to determine the SNR. The monitoring device may compare the SNR to an SNR threshold, and determine based on the comparison whether connection with the remote device is interrupted or lost. For example, the monitoring device may determine that the communication connection with the remote device is interrupted or lost based on a determination that the SNR is less than the SNR threshold.

At block 405, in some implementations, the monitoring device may be configured to implement a keep-alive mechanism, in which wireless signals are sent (e.g., periodically) between the monitoring device and remote device to confirm whether connection between the devices if still active. For example, the monitoring device may periodically ping the remote device with a keep-alive signal to ensure the server is reachable. If the monitoring device does not receive a response (e.g., an acknowledgement signal) within a certain period of time (e.g., a timeout), the monitoring device may determine that connection with the remote device is interrupted or lost. In some examples, at block 405, after the monitoring device transmits data (e.g., physiological data originating from a sensor) to a remote device, the remote device may send an acknowledgement signal to the monitoring device. If the remote device does not detect the acknowledgement signal (e.g., the acknowledgement signal has not been received by the monitoring device from the remote device), or does not detect the acknowledgement signal within a certain period of time, the monitoring device may determine that connection with the remote device has been interrupted or lost.

In some implementations, at block 405, the monitoring device may be configured to monitor error rates or packet loss. For example, the monitoring device may monitor for packet retransmissions or error rates in received packets. High numbers of retransmissions and/or high error rates can be indicative of interrupted or lost connection between the monitoring device and the remote device. The monitoring device may compare determined error rates or packet loss to a predetermined threshold, and determine based on the comparison whether connection with the remote device is interrupted or lost. For example, the monitoring device may determine that the communication connection with the remote device is interrupted or lost based on a determination that the error rate or packet loss is above a predetermined threshold.

At block 407, responsive to a determination that communication connection with the remote device is interrupted or lost, the monitoring device may determine a physiological parameter based on the received physiological data, such as data originating from one or more physiological sensors. The monitoring device may determine a physiological parameter based on received physiological signals, such as signal originating from one or more physiological signals. The determined physiological parameters can include any available type of physiological parameter, such as any of the physiological parameters described herein. For example, determined physiological parameters can include, but are not limited to, heart rate (or pulse rate), respiration rate, blood pressure, blood oxygen saturation, hemoglobin content, VO2 max, temperature, and the like.

At block 409, the monitoring device can determine whether the physiological parameter satisfies an alarm condition, such as a physiological threshold condition. The monitoring device may determine whether one or more physiological parameters satisfy one or more alarm conditions, such as according to one or more physiological threshold conditions (e.g., distinct alarm conditions for each physiological parameter). The physiological threshold condition can include limits such as lower and upper limits. In some implementations, the monitoring device can determine a change in the physiological parameter between a first time and a second time. For example, the monitoring device may combine physiological data with physiological parameter time instances and determine a difference between beginning and ending timestamps associated with a physiological parameter. In some implementations, the monitoring device can determine whether the physiological parameter satisfies a physiological threshold condition and/or whether the physiological parameter satisfies a physiological threshold condition more than a certain number of times. In some implementations, the monitoring device can determine whether a value associated with the physiological parameter satisfies a physiological threshold condition. In some implementations, the monitoring device can determine whether a change in the physiological parameter satisfies a physiological threshold condition. In some implementations, the monitoring device can determine whether a rate of change of the physiological parameter satisfies a physiological threshold condition. In some implementations, the monitoring device can determine whether a change in the rate of change of the physiological parameter satisfies a physiological threshold condition. In some implementations, the monitoring device can determine whether the physiological parameter is changing in a certain direction (e.g., positive change or negative change). In some implementations, the monitoring device may make a determination based on any combination of the foregoing examples.

At block 409, in some implementations, the physiological threshold condition(s) may be fixed or may be programmable. For example, the physiological threshold condition(s) may be preloaded and stored in a local memory of the monitoring device and accessible by one or more hardware processors. In some implementations, the physiological threshold condition(s) can be set and/or modified by a user utilizing a computing device (e.g., via a smartphone application, a mobile application, and/or a web application executing in a browser) in communication with the monitoring device. In some implementations, the physiological threshold conditions(s) may be based at least in part on a history of sensor data such as relating to an average of historical sensor data. In some implementations, at block 409, the physiological threshold condition(s) can be based on alarm configuration data receive from the remote device.

In some implementations, the physiological threshold condition(s) used by the monitoring device may be different than the physical threshold condition(s) used by the remote device. For example, the monitoring device and remote device may generate alarms under different physiological conditions, such as based on different physiological conditions associated with the same physiological parameter. The remote device may implement a complex alarm algorithm, such as described herein, that intakes multiple different physiological inputs and/or combinations of inputs from multiple different monitoring devices, as well as intakes contextual information, whereas the remote device may implement a simple alarm algorithm that intakes, for example, a single physiological input for comparison to a simple alarm threshold limit. The simple alarm threshold limit can be a predetermined physiological threshold associated with a physiological parameter. In some implementations, at block 409, the predetermined threshold may be preloaded in local memory of the monitoring device or can be based on alarm configuration data received from the remote device, such as prior to interruption or loss of connection with the remote device. The alarm configuration data may be indicative of one or more physiological thresholds associated with one or more physiological parameters, respectively.

At block 409, in some implementations, the monitoring device may compare the determined physiological parameter to a physiological alarm condition, such as a predetermined threshold, and determine based on the comparison whether the physiological parameter satisfies the alarm condition. For example, the monitoring device may determine that the physiological parameter satisfies the alarm condition based on a determination that the physiological parameter is above or below a predetermined threshold, and/or is outside of a predetermined threshold range.

In some implementations, at block 409, the monitoring device may use the physiological threshold condition (e.g., the predetermined physiological threshold) for generating alarms only when connection between the monitoring device and the remote device is interrupted or lost. For example, the monitoring device may compare the physiological parameter to the predetermined threshold only when connection to the remote device is interrupted or lost.

At block 411, responsive to a determination that the physiological parameter satisfies a physiological threshold condition, the monitoring device can generate an alarm. In some implementations, at block 411, the monitoring device may generate the alarm only if connection with the remote device is interrupted or lost. The alarm can include a visual, auditory, and/or haptic alarm. For example, the alarm can include visual, auditory, and/or haptic feedback.

In some implementations, at block 411, the monitoring device may output the alarm. For example, the monitoring device may render and cause visual indicia associated with the alarm to be displayed in a display of the monitoring device. In some examples, the monitoring device may output a sound associated with the alarm from a speaker of the monitoring device. In some examples, the monitoring device (or portions thereof) may vibrate. In some implementations, the monitoring device may cause output of the alarm by another device, such as another computing device. The monitoring device may send alarm signals indicative of the generated alarm to another device such that the other device outputs the alarm. For example, the monitoring device may output the alarm to a display of a display terminal (the display terminal, based on the alarm signal, may render and display visual indicia of the alarm). In some examples, the monitoring device may output the alarm to a loudspeaker such that the loudspeaker plays a sound associated with the alarm.

### Additional Implementations

As used herein, “system,” “instrument,” “apparatus,” and “device” generally encompass both the hardware (for example, mechanical and electronic) and, in some implementations, associated software (for example, specialized computer programs for graphics control) components.

It is to be understood that not necessarily all objects or advantages may be achieved in accordance with any particular implementation described herein. Thus, for example, those skilled in the art will recognize that certain implementations may be configured to operate in a manner that achieves or optimizes one advantage or group of advantages as taught herein without necessarily achieving other objects or advantages as may be taught or suggested herein.

Each of the processes, methods, and algorithms described in the preceding sections may be embodied in, and fully or partially automated by, code modules executed by one or more computer systems or computer processors including computer hardware. The code modules may be stored on any type of non-transitory computer-readable medium or computer storage component, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

Many other variations than those described herein will be apparent from this disclosure. For example, depending on the implementation, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the implementations disclosed herein can be implemented as electronic hardware, computer software, or combinations of both. To clearly illustrate this interchangeability of hardware and software, various illustrative components, blocks, modules, and elements have been described herein generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various features and processes described herein may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example implementations. The example systems and components described herein may be configured differently than described. For example, elements may be added to, removed from, or rearranged compared to the disclosed example implementations.

The various illustrative logical blocks and modules described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can include electrical circuitry configured to process computer-executable instructions. In another implementation, a processor includes an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the implementations disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” mechanism one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, and so forth, may be either X, Y, or Z, or any combination thereof (for example, X, Y, and/or Z). Thus, such disjunctive language is not generally intended to, and should not, imply that certain implementations require at least one of X, at least one of Y, or at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the implementations described herein in which elements or functions may be deleted, executed out of order from that shown or discussed, including substantially concurrently or in reverse order, depending on the functionality involved, as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

All of the methods and processes described herein may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the herein-described implementations, the elements of which are to be understood as being among other acceptable examples. All such modifications and variations are intended to be included herein within the scope of this disclosure. The section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

1. A physiological monitoring system:
a remote device comprising one or more hardware processors configured to:
receive physiological data from a noninvasive medical monitoring device;
process the physiological data using a first alarm algorithm based on a plurality of physiological inputs to determine whether a first alarm condition is satisfied;
generate and transmit an alarm to the noninvasive medical monitoring device if the first alarm condition is satisfied; and
transmit alarm configuration data specifying at least one second alarm condition to the noninvasive medical monitoring device; and
the noninvasive medical monitoring device comprising one or more hardware processors in communication with a physiological sensor and a communication component, the one or more hardware processors configured to:
store the alarm configuration data received from the remote device in local memory;
determine whether a connection with the remote device is lost;
responsive to a determination that the connection with the remote device is lost, process physiological data using a second alarm algorithm based on the stored alarm configuration data to determine whether the second alarm condition is satisfied, and generate a local alarm only if the connection with the remote device is lost and the second alarm condition is satisfied;
wherein the second alarm algorithm is less computationally intensive than the first alarm algorithm, and
wherein, when the connection with the remote device is not lost, the noninvasive medical monitoring device is configured to rely on the remote device to generate alarms.

2. The system of claim 1, wherein the first alarm algorithm executed by the remote device comprises analysis of multiple physiological parameters, historical trends, or contextual patient information.

3. The system of claim 1, wherein the second alarm algorithm executed by the noninvasive medical monitoring device comprises comparison of a physiological parameter to a predetermined threshold.

4. The system of claim 1, wherein the noninvasive medical monitoring device is further configured to buffer physiological data generated during a period in which the connection with the remote device is lost, and to transmit the buffered physiological data to the remote device when the connection is reestablished.

5. The system of claim 1, wherein the remote device is further configured to update the alarm configuration data and transmit updated alarm configuration data to the noninvasive medical monitoring device.

6. The system of claim 1, wherein the first alarm algorithm executed by the remote device determines the first alarm condition based on a combination of a threshold or threshold range for a physiological parameter and at least one of: patient measurement history, patient comorbidity indications, measurements from other physiological parameters, types of procedures occurring, treatments administered or indicated, or other risk factors.

7. The system of claim 1, wherein the alarm configuration data transmitted from the remote device to the noninvasive medical monitoring device comprises at least one of: a threshold value, a threshold range, a time duration, a rate of change, or a combination thereof.

8. The system of claim 1, wherein the noninvasive medical monitoring device is further configured to output the local alarm as at least one of a visual, auditory, or haptic signal.

9. The system of claim 1, wherein the determination that the connection with the remote device is lost is based on at least one of: a signal strength condition, a failure to receive an acknowledgement signal, or a timeout period without receiving wireless signals from the remote device.

10. The system of claim 1, wherein the noninvasive medical monitoring device is further configured to receive updated alarm configuration data from the remote device and to overwrite a previously stored alarm configuration data in local memory.

11. The system of claim 1, wherein the remote device is further configured to, upon reestablishing communication with the noninvasive medical monitoring device, receive buffered physiological data and reprocess the buffered data using the first alarm algorithm to determine whether any first alarm conditions would have been satisfied during a period of lost connection.

12. The system of claim 1, wherein the noninvasive medical monitoring device is further configured to store a log of all local alarms generated during a period of lost connection and to transmit the log to the remote device when the connection is reestablished.

13. The system of claim 1, wherein the remote device is further configured to generate a report indicating any discrepancies between alarms generated by the noninvasive medical monitoring device during a period of lost connection and alarms that would have been generated by the first alarm algorithm.

14. The system of claim 1, wherein the noninvasive medical monitoring device is further configured to display an indication of connection status with the remote device.

15. The system of claim 1, wherein the alarm configuration data comprises patient-specific alarm thresholds determined by the remote device based on analysis of historical physiological data.

16. A noninvasive medical monitoring device, the medical device comprising:
one or more hardware processors in communication with a communication component, the communication component configured to operate one or more wireless communication protocols, the communication component configured to permit wireless connection between the monitoring device and a remote device such that the monitoring device sends and receives, to and from the remote device, wireless signals;
the one or more hardware processors in communication with a physiological sensor; and
the one or more hardware processors configured to:
determine whether the connection with the remote device is lost;
responsive to a determination that the connection with the remote device is lost, determine at least one physiological parameter based on the at least one physiological signal;
determine whether the physiological parameter satisfies a predetermined alarm condition; and
responsive to the determination that the physiological parameter satisfies the predetermined alarm condition, generate an alarm.

17. (canceled)

18. The noninvasive medical monitoring device of claim 16, wherein the one or more hardware processors are configured to generate the alarm only if connection to the remote device is lost.

19. (canceled)

20. (canceled)

21. The noninvasive medical monitoring device of claim 16, wherein the one or more hardware processors are configured to receive, from the remote device, alarm configuration data that specifies the predetermined alarm condition that is used by the monitoring device for generating the alarm if connection to the remote device is lost.

22. (canceled)

23. The noninvasive medical monitoring device of claim 16, wherein the one or more hardware processors are further configured to output the alarm to a display of a display terminal, a display of the monitoring device, or to a loudspeaker.

24. (canceled)

25. (canceled)

26. (canceled)

27. (canceled)

28. (canceled)

29. (canceled)

30. The noninvasive medical monitoring device of claim 16, wherein physiological data associated with the at least one physiological signal is stored in a buffer until connection with the remote device is reestablished.

31-76. (canceled)
